(19) United States
(12) Patent Application Publication
(10) Pub. No.: US 2024/0424207 A1
(43) Pub. Date: Dec. 26, 2024

(54) CLOSED-LOOP ARTIFICIAL PANCREAS INSULIN INFUSION CONTROL SYSTEM

(71) Applicant: MEDTRUM TECHNOLOGIES INC., Shanghai (CN)

(72) Inventor: Cuijun Yang, Shanghai (CN)

(73) Assignee: MEDTRUM TECHNOLOGIES INC., Shanghai (CN)

(21) Appl. No.: 18/703,368

(22) PCT Filed: Oct. 25, 2021
(86) PCT No.: PCT/CN2021/126005

§ 371 Date: Apr. 22, 2024

(51) Int. Cl. A61M 5/172; G16H 20/17

(52) U.S. Cl. CPC A61M 5/1723; G16H 20/17; A61M 2230/201

(57) ABSTRACT

A closed-loop artificial pancreas insulin infusion control system is provided. The system includes a detection module configured to detect the current blood glucose level continuously, a program module preset with an rMPC algorithm converting the asymmetric blood glucose in the original physical space to the approximately symmetric blood glucose risk in the risk space and target blood glucose level, wherein the rMPC algorithm calculates insulin infusion instructions based on blood glucose risk, and an infusion module connected to the program module and controlled by the program module to infuse insulin according to the corresponding output instructions calculated by the rMPC algorithm. The system is preset with a rMPC algorithm, which converts the asymmetric blood glucose in the original physical space to the approximately symmetric blood glucose risk in the risk space, the recent changes of blood glucose and the asymmetry of distribution are taken into account in the rMPC algorithm.

### TECHNICAL FIELD

The present invention mainly relates to the field of medical device, and in particular, to a closed-loop artificial pancreas insulin infusion control system.

### BACKGROUND

The pancreas of healthy people can automatically secrete the required insulin/glucagon according to the glucose level in the human blood, thereby maintaining a reasonable range of blood glucose fluctuations. However, for diabetic patients, the function of their pancreas has been severely compromised, and the pancreas cannot secrete the required dosage of insulin. Therefore, diabetes mellitus is defined as a metabolic disease caused by abnormal pancreatic function, and it is also classified as one of the top three chronic conditions by the WHO. The present medical advancement has not been able to find a cure for diabetes mellitus. Yet, the best the technology could do is control the onset symptoms and complications by stabilizing the blood glucose level for diabetes patients.

Diabetic patients on an insulin pump need to check their blood glucose before infusing insulin into their bodies. At present, most detection methods can continuously detect blood glucose and send the blood glucose data to the remote device in real-time for the user to view. This detection method is called Continuous Glucose Monitoring (CGM), which requires the detection device to be attached to the surface of the patients skin, and the sensor carried by the device to be inserted into the interstitial fluid for testing. According to the blood glucose (BG) level, the infusion system mimics an artificial pancreas to fill the gaps of the required insulin amount via the closed-loop pathway or the semi-closed-loop pathway.

At present, in order to achieve insulin infusion controlled by closed-loop or semi-closed-loop, the model-predict-control (MPC) algorithm uses predictive models to predict the future behavior of the insulin pump's output under the changes of blood glucose. It can casily handle additional inputs, such as meals, exercise, etc., and its model parameters have clear physiological meanings, which is convenient for personalization and optimization, so that MPC algorithm has been is widely researched. While MPC algorithm faces the dilemma of establishing an accurate model and dealing with large computations, which may lead to deviation for the predicted infusion.

Therefore, in the prior art, there is an urgent need for a closed-loop artificial pancreas insulin infusion control system with optimized MPC algorithm.

### BRIEF SUMMARY OF THE INVENTION

The embodiment of the present invention discloses a closed-loop artificial pancreas insulin infusion control system. The system is preset with a rMPC algorithm, which converts the asymmetric blood glucose in the original physical space to the approximately symmetric blood glucose risk in the risk space, the recent changes of blood glucose and the asymmetry of distribution are taken into account in the rMPC algorithm, giving the rMPC algorithm the advantages of precision and flexibility, realizing precise control for closed-loop artificial pancreas insulin infusion system.

The invention discloses a closed-loop artificial pancreas insulin infusion control system, including: a detection module configured to detect the current blood glucose level G continuously; a program module, preset with an rMPC algorithm that converts the asymmetric blood glucose in the original physical space to the approximately symmetric blood glucose risk in the risk space and target blood glucose level G_{B}, the rMPC algorithm calculates insulin infusion instructions based on blood glucose risk; and an infusion module, connected to the program module, and is controlled by the program module to infuse insulin according to the corresponding output instructions calculated by the rMPC algorithm.

According to one aspect of the present invention, the rMPC algorithm consists of the prediction model, the value function and the constraints, where the prediction model is:

$${x}_{t + 1} = A {x}_{t} + {BI}_{r}$$

$${G}_{t} = C {x}_{t}$$

Where:

$${x}_{t + 1} = [ \begin{matrix}{G}_{t + 1} \\ {G}_{t} \\ {G}_{t ‐ 1}\end{matrix} ] ;$$

xₜ₊₁ represents the state parameter at the next moment,

$${x}_{t} = [ \begin{matrix}G \\ {G}_{t - 1} \\ {G}_{t - 2}\end{matrix} ] ;$$

xₜ represents the current state parameter,
Iₜ represents the amount of insulin infusion at the current moment;
Gₜ represents the blood glucose concentration at the current moment.
The parameter matrix is as follows:

$$A = [ \begin{matrix}{b}_{1} & {b}_{2} & {b}_{3} \\ 1 & 0 & 0 \\ 0 & 1 & 0\end{matrix} ]$$

$$B = [ \begin{matrix}{k}_{i} \\ 0 \\ 0\end{matrix} ]$$

$$C = [ \begin{matrix}1 & 0 & 0\end{matrix} ]$$

Where:
b1, b2, b3, Ki are prior values.
the value function is:

$${J}^{r M P C} = {\sum }_{j = 1}^{p} {‖ {r}_{t + j} ‖}^{2} + R {\sum }_{j = 1}^{N} {‖ {I}_{t + j}^{′} ‖}^{2}$$

Where:
rₜ₊ⱼ represents the blood glucose risk index after step j;
I′ₜ₊ⱼ represents the change of insulin infusion after step j.
t represents the current moment;
N and P are the number of steps in the control time window and the predictive time window respectively;
R is the weighting coefficient of the insulin component.

According to one aspect of the present invention, the blood glucose risk index rₜ₊ⱼ is calculated as:

$$\{ \begin{matrix}{r}_{t + j} = ( {G}_{t + j} - {G}_{B} ) , if {G}_{B} < {G}_{t + j} \leq 180 mg / dL \\ {r}_{t + j} = ( {G}_{t + j} - {G}_{B} ) * 0.5 , if 180 < {G}_{t + j} \leq 300 mg / dL \\ {r}_{t + j} = ( {G}_{t + j} - {G}_{B} ) * 0.2 , if 300 < {G}_{t + j} \leq 400 mg / dL \\ {r}_{t + j} = ( 400 - {G}_{B} ) * 0.2 , if {G}_{t + j} > 400 mg / dL\end{matrix}$$

where:
Gₜ₊ⱼ represents the blood glucose level detected in step j.

According to one aspect of the present invention, the blood glucose risk index rₜ₊ⱼ is calculated as:

$$\{ \begin{matrix}{r}_{t + j} = {G}_{t + j} - {G}_{B} , if {G}_{t + j} \leq {G}_{B} \\ {r}_{t + j} = 100 * \frac{{G}_{t + j} - {G}_{B}}{{G}_{t + j}} , if {G}_{t + j} > {G}_{B}\end{matrix}$$

Where:
Gₜ₊ⱼ represents the blood glucose level detected in step j.

According to one aspect of the present invention, the blood glucose risk index rₜ₊ⱼ is calculated as:

$$\{ \begin{matrix}{r}_{t + j} = - r ( {G}_{t + j} ) , if {G}_{t + j} \leq {G}_{B} \\ {r}_{t + j} = r ( {G}_{t + j} ) , if {G}_{t + j} > {G}_{B}\end{matrix}$$

Where:

$$r ( {G}_{t + j} ) = 10 * {f ( {G}_{t + j} )}^{2}$$

The conversion function f(Gₜ₊ⱼ) is as follows:

$$f ( {G}_{t + j} ) = 1.509 * [ {( ln ( {G}_{t + j} ) )}^{1.084} - 5.381 ]$$

Where:
Gₜ₊ⱼ represents the blood glucose level detected in step j.

According to one aspect of the present invention, the blood glucose risk index rₜ₊ⱼ is calculated as:

$$\{ \begin{matrix}{r}_{t + j} = {G}_{t + j} - {G}_{B} , if {G}_{t + j} \leq {G}_{B} \\ {r}_{t + j} = - 4.8265 * 1 {0}^{4} - 4 * {G}_{t + j}^{2} + 0.45563 * {G}_{t + j} - 44.8 \; 55 , if {G}_{t + j} > {G}_{B}\end{matrix}$$

Where:
Gₜ₊ⱼ represents the blood glucose level detected in step j.

According to one aspect of the present invention, the maximum value of the blood glucose risk index rₜ₊ⱼ is limited as: |rₜ₊ⱼ|=min (rₜ₊ⱼ|, n).

According to one aspect of the present invention, the range of the limit of the maximum value n is from 0 to 80 mg/dL.

According to one aspect of the present invention, the value of n is 60 mg/dL.

According to one aspect of the present invention, When the detected blood glucose concentration in step j Gₜ₊ⱼ is less than G_{B}, the BGRI method is used, the blood glucose risk index rₜ₊ⱼ is calculated as:

$${r}_{t + j} = r ( {G}_{t + j} ) , if {G}_{t + j} > {G}_{B}$$

Where:

$$r ( {G}_{t + j} ) = 10 * {f ( {G}_{t + j} )}^{2}$$

The conversion function f(Gₜ₊ⱼ) is as follows:

$$f ( {G}_{t + j} ) = 1.509 * [ {( ln ( {G}_{t + j} ) )}^{1.084} - 5.381 ]$$

when the detected blood glucose concentration in step j Gₜ₊ⱼ is greater than G_{B}, the CVGA method is used, the blood glucose risk index rₜ₊ⱼ is calculated as:

$${r}_{t + j} = {G}_{t + j} - {G}_{B} {G}_{t + j} \leq {G}_{B}$$

Where:
Gₜ₊ⱼ represents the blood glucose level detected in step j.

According to one aspect of the present invention, the maximum value of the blood glucose risk index rₜ₊ⱼ is limited as: |rₜ₊ⱼ|=min (|rₜ₊ⱼ|, n).

According to one aspect of the present invention, the range of the limit of the maximum value n is from 0 to 80 mg/dL.

According to one aspect of the present invention, the value of n is 60 mg/dL.

According to one aspect of the present invention, when the detected blood glucose concentration in step j Gₜ₊ⱼ is less than G_{B}, the BGRI method is used, the blood glucose risk index rₜ₊ⱼ is calculated as:

$${r}_{t + j} = - r ( {G}_{t + j} ) , if {G}_{t + j} \leq {G}_{B}$$

Where:

$$r ( {G}_{t + j} ) = 10 * {f ( {G}_{t + j} )}^{2}$$

the conversion function f(Gₜ₊ⱼ) is as follows:

$$f ( {G}_{t + j} ) = 1.509 * [ {( ln ( {G}_{t + j} ) )}^{1.084} - 5.381 ]$$

when the detected blood glucose concentration in step j Gₜ₊ⱼ is great than G_{B}, the segmented weighting converting is used, the blood glucose risk index rₜ₊ⱼ is calculated as:

$${r}_{t + j} = - 4.8265 * 1 {0}^{4} - 4 * {G}_{t + {j}^{2}} + 0.45563 * {G}_{t + j} - 44 \text{.855} , if {G}_{t + j} > {G}_{B}$$

where:
Gₜ₊ⱼ represents the blood glucose level detected in step j.

According to one aspect of the present invention, when the detected blood glucose concentration in step j Gₜ₊ⱼ is less than G_{B}, the BGRI method is used, the blood glucose risk index rₜ₊ⱼ is calculated as:

$${r}_{t + j} = - r ( {G}_{t + j} ) , if {G}_{t + j} \leq {G}_{B}$$

Where:

$$r ( {G}_{t + j} ) = 10 * {f ( {G}_{t + j} )}^{2}$$

The conversion function f(Gₜ₊ⱼ) is as follows:

$$f ( {G}_{t + j} ) = 1.509 * [ {( ln ( {G}_{t + j} ) )}^{1.084} - 5.381 ]$$

when the detected blood glucose concentration in step j Gₜ₊ⱼ is great than G_{B}, the segmented weighting converting is used, the blood glucose risk index rₜ₊ⱼ is calculated as:

$$\{ \begin{matrix}{r}_{t + j} = ( {G}_{t + j} - {G}_{B} ) , if {G}_{B} < {G}_{t + j} \leq 180 mg / dL \\ {r}_{t + j} = ( {G}_{t + j} - {G}_{B} ) * 0.5 , if 180 < {G}_{t + j} \leq 300 mg / dL \\ {r}_{t + j} = ( {G}_{t + j} - {G}_{B} ) * 0.2 , if 300 < {G}_{t + j} \leq 400 mg / dL \\ {r}_{t + j} = ( 400 - {G}_{B} ) * 0.2 , if {G}_{t + j} > 400 mg / dL\end{matrix}$$

Where:
Gₜ₊ⱼ represents the blood glucose level detected in step j.

According to one aspect of the present invention, when the detected blood glucose concentration in step j Gₜ₊ⱼ is less than G_{B}, the BGRI method is used, the blood glucose risk index rₜ₊ⱼ is calculated as:

$${r}_{t + j} = - r ( {G}_{t + j} ) , if {G}_{t + j} \leq {G}_{B}$$

Where:

$$r ( {G}_{t + j} ) = 10 * {f ( {G}_{t + j} )}^{2}$$

The conversion function f(Gₜ₊ⱼ) is as follows:

$$f ( {G}_{t + j} ) = 1.509 * [ {( ln ( {G}_{t + j} ) )}^{1.084} - 5.381 ]$$

when the detected blood glucose concentration in step j Gₜ₊ⱼ is great than G_{B}, the segmented weighting converting is used, the blood glucose risk index rₜ₊ⱼ is calculated as:

$${r}_{t + j} = 100 * \frac{{G}_{t + j} - {G}_{B}}{{G}_{t + j}} , {G}_{t + j} > {G}_{B}$$

Where:
Gₜ₊ⱼ represents the blood glucose level detected in step j.

According to one aspect of the present invention, the target blood glucose value G_{B} is 80-140 mg/dL.

According to one aspect of the present invention, the target blood glucose value G_{B} is 110-120 mg/dL.

According to one aspect of the present invention, the rMPC algorithm also includes one or more of the following processing methods:
\textcircled{1} according to the insulin absorption delay in the artificial pancreas control system, the amount of plasma insulin that is not absorbed in the body γ [symbol] (t+j) is deducted.

$${rI}_{t + j}^{′} = {rI}_{t + j} - IOB ( t + j )$$

Where:
Iₜ₊ⱼ represents the infusion instruction sent to the insulin infusion system after step j;
rI_{c(t+i)} represents the infusion instruction with compensation sent to the insulin infusion system after step j;
γ represents the compensation coefficient of the estimated plasma insulin concentration to the algorithm output.
[symbol] (t+j) represents the estimation of plasma insulin concentration in step j.
\textcircled{2} according to the delayed of insulin onset in the artificial pancreas control system, the amount of insulin that has not yet worked in the body IOB(t+j) is deducted:

$${rI}_{t + j}^{′} = {rI}_{t + j} - IOB ( t + j )$$

Where:
rI′ₜ₊ⱼ represents the infusion instruction sent to the insulin infusion system after deducting IOB at step j after risk conversion;
rIₜ₊ⱼ represents the infusion instruction sent to the insulin infusion system at step j after risk conversion;
IOB(t+j) represents the amount of insulin that has not yet worked in the body at time t+j.
\textcircled{3} the autoregressive method is used to compensate for the detecting delay of blood glucose concentration and interstitial fluid glucose concentration.

According to one aspect of the present invention, the estimation of plasma insulin concentration in step j [symbol] (t+j) is obtained by autoregressive method.

According to one aspect of the present invention, the range of γ is 0.4-0.6.

According to one aspect of the present invention, γ is 0.5.

According to one aspect of the present invention, the amount of insulin that has not yet worked in the body at time t+j IOB(t+j) is obtained from IOB curves.

According to one aspect of the present invention, the amount of insulin that has not yet worked in the body at time t+j IOB(t+j) is divided in to meal insulin and non-meal insulin;

$$IOB ( t + j ) = {IOB}_{m , t + j} + {IOB}_{o , t + j}$$

where:

$$\{ \begin{matrix}{IOB}_{m , t + j} = {D}_{2} {I}_{m , t + j} \\ {IOB}_{o , t + j} = {D}_{2} {I}_{o , t + j} if {G}_{t + j} > 300 mg / dL \\ {IOB}_{o , t + j} = {D}_{4} {I}_{o , t + j} if 300 mg / dL \geq {G}_{t + j} > 200 mg / dL \\ {IOB}_{o , t + j} = {D}_{6} {I}_{o , t + j} if 200 mg / dL \geq {G}_{t + j} > 140 mg / dL \\ {IOB}_{o , t + j} = {D}_{8} {I}_{o , t + j} if {G}_{t + j} \leq 140 mg / dL\end{matrix}$$

where:
   IOB_{m,t+j} represents the amount of meal insulin that has not yet worked in the body at time t+j;
   IOB_{o,t+j} represents the amount of non-meal insulin that has not yet worked in the body at time t+j;
   Di(i=2-8) represents the respective coefficients corresponding to the IOB curve with insulin action time i;
   I_{m,t+j} represents the amount of meal insulin at time t+j;
   I_{o,t+j} represents the amount of non-meal insulin at time t+j;
   IOB(t+j) represents the amount of insulin that has not yet worked in the body at time t+j

According to one aspect of the present invention, any two of the detection module, the program module and the infusion module are connected to each other configured to form a single part whose attached position on the skin is different from the third module.

According to one aspect of the present invention, the detection module, the program module and the infusion module are connected together configured to form a single part which is attached on only one position on the skin.

Compared with the prior art, the technical solution of the present invention has the following advantages:

In the closed-loop artificial pancreas insulin infusion control system disclosed in the present invention, the preset rMPC algorithm, which converts the asymmetric blood glucose in the original physical space to the approximately symmetric blood glucose risk in the risk space, the recent changes of blood glucose and the asymmetry of distribution are taken into account in the rMPC algorithm, giving the rMPC algorithm the advantages of precision and flexibility, realizing precise control for closed-loop artificial pancreas insulin infusion system.

Furthermore, the rMPC algorithm can be processed separately or combined with segmented weighting method, relative value method, BRGI method and improved CVGA method, and can flexibly select target blood glucose concentration or zero-risk point blood glucose concentration or equal-risk point data pairs according to the actual situation to make rMPC algorithm more stable, and still has slow adjustment ability in a relatively flat interval, so that the closed-loop artificial pancreas can face more complicated use scenarios, so as to achieve more accurate blood sugar control.

Furthermore, the rMPC algorithm also compensates for insulin absorption delay, insulin onset delay, and interstitial fluid glucose concentration and blood glucose detecting delay, making the output calculated by the rMPC algorithm more reliable.

Furthermore, in order to compensate for the insulin onset delay, the IOB is divided into meal insulin and non-meal insulin in the rMPC algorithm, which can make insulin being cleared faster when meals ingesting or blood glucose are too high, and can obtain greater insulin output and regulate blood glucose more quickly. When approaching the target, a longer insulin action time curve is used to make insulin being clear ed more slowly, and blood sugar regulation is more conservative and stable.

Furthermore, the detection module, the program module and the infusion module are connected together configured to form a single part which is attached on only one position on the skin. If the three modules are connected as a whole and attached in the only one position, the number of the device on the user skin will be reduced, thereby reducing the interference of more attached devices on user activities. At the same time, it also effectively solves the problem of the poor wireless communication between separating devices, further enhancing the user experience.

### BRIEF DESCRIPTION OF THE DRAWINGS

FIG. 1 is a schematic diagram of the module relationship of the closed-loop artificial pancreas insulin infusion control system according to one embodiment of the present invention.

FIG. 2 is a comparison diagram of the blood glucose in the original physical space and the risk space, which is obtained through the segmented weighting and the relative value conversion according to an embodiment of the present invention.

FIG. 3 is a comparison diagram of the blood glucose in the original physical space and the risk space, which is obtained through the BGRI and CVGA method according to an embodiment of the present invention.

FIG. 4 is a schematic diagram of an insulin IOB curve according to an embodiment of the present invention.

FIG. 5 is a schematic diagram of four types of mainstream clinical optimal basal rate settings according to an embodiment of the present invention

FIG. 6 is a schematic diagram of the module relationship of the closed-loop artificial pancreas insulin infusion control system according to another embodiment of the present invention.

FIG. 7 is a schematic diagram of the module relationship of the closed-loop artificial pancreas insulin infusion control system according to another embodiment of the present invention.

FIG. 8 is a schematic diagram of the module relationship of the closed-loop artificial pancreas multi-drug infusion control system according to another embodiment of the present invention.

FIG. 9 is a schematic diagram of dual-drug switching according to an embodiment of the present invention.

FIG. 10 is a schematic diagram of the module relationship of the closed-loop artificial pancreas insulin infusion control system according to another embodiment of the present invention.

### DETAILED DESCRIPTION

As mentioned above, due to the classic MPC algorithm faces the dilemma of establishing an accurate model and dealing with large computations, which may lead to deviation for the predicted infusion.

In order to solve this problem, the present invention provides a closed-loop artificial pancreas insulin infusion control system, the system is preset with a rMPC algorithm, which converts the asymmetric blood glucose in the original physical space to the approximately symmetric blood glucose risk in the risk space, the recent changes of blood glucose and the asymmetry of distribution are taken into account in the rMPC algorithm, giving the rMPC algorithm the advantages of precision and flexibility, realizing precise control for closed-loop artificial pancreas insulin infusion system.

Various exemplary embodiments of the present invention will now be described in detail with reference to the drawings. The relative arrangement of the components and the steps, numerical expressions and numerical values set forth in the embodiments are not to be construed as limiting the scope of the invention.

In addition, it should be understood that, for case of description, the dimensions of the various components shown in the figures are not necessarily drawn in the actual scale relationship, for example, the thickness, width, length or distance of certain units may be exaggerated relative to other parts.

The following description of the exemplary embodiments is merely illustrative, and is not intended to be in any way limiting the invention and its application or use. The techniques, methods, and devices that are known to those of ordinary skill in the art may not be discussed in detail, but such techniques, methods, and devices should be considered as part of the specification.

It should be noted that similar reference numerals and letters indicate similar items in the following figures. Therefore, once an item is defined or illustrated in a drawing, it will not be discussed further in the following description of the drawings.

FIG. 1 is a schematic diagram of the module relationship of the closed-loop artificial pancreas insulin infusion control system according to the embodiment of the present invention.

The closed-loop artificial pancreas insulin infusion control system disclosed in the embodiment of the present invention mainly includes a detection module 100, a program module 101, and an infusion module 102.

The detection module 100 is used to continuously detect the user's real-time blood glucose (BG) level. Generally, detection module 100 is a Continuous Glucose Monitoring (CGM) for detecting real-time BG, monitoring BG changes, and sending them to the program module 101.

Program module 101 is used to control the detection module 100 and the infusion module 102. Therefore, program module 101 is connected to detection module 100 and infusion module 102, respectively. Here, the connection refers to a conventional electrical connection or a wireless connection.

The infusion module 102 includes the essential mechanical assemblies used to infuse insulin and is controlled by program module 101. According to the current insulin infusion dose calculated by program module 101, infusion module 102 injects the current insulin dose required into the user's body. At the same time, the real-time infusion status of infusion module 102 can also be fed back to program module 101.

The embodiment of the present invention does not limit the specific positions and connection relationships of the detection module 100, the program module 101 and the infusion module 102, as long as the aforementioned functional conditions can be satisfied.

As in an embodiment of the present invention, the three are electrically connected to form a single part. Therefore, the three modules can be attached on only one position of the user's skin. If the three modules are connected as a whole and attached in only one position, the number of the device on the user skin will be reduced, thereby reducing the interference of more attached devices on user activities. At the same time, it also effectively solves the problem of poor wireless communication between separating devices, further enhancing the user experience.

Another embodiment of the present invention is that the program module 101 and the infusion module 102 are electrically connected to form a single part, while the detection module 100 is separately provided in another part. At this time, the detection module 100 and the program module 101 transmit wireless signals to realise the mutual connection. Therefore, program module 101 and infusion module 102 can be attached to the user's skin position while the detection module 100 is attached to the other position.

Another embodiment of the present invention is that the program module 101 and the detection module 100 are electrically connected, forming a single part, while the infusion module 102 is separately provided in another part. The infusion module 102 and the program module 101 transmit wireless signals to realise the mutual connection. Therefore, program module 101 and the detection module 100 can be attached to the same position of the user's skin while the infusion module 102 is attached to the other position.

Another embodiment of the present invention is that the three are provided in different parts, thus being attached to different positions. Simultaneously, program module 101, detection module 100, and infusion module 102 transmit wireless signals to realize the mutual connection.

It should be noted that the program module 101 of the embodiment of the present invention also has functions such as storage, recording, and access to the database. Thus, program module 101 can be reused. In this way, the user's physical condition data can be stored, but the production and consumption costs can be saved. As described above, when the service life of the detection module 100 or the infusion module 102 expires, program module 101 can be separated from the detection module 100, the infusion module 102, or both the detection module 100 and the infusion module 102.

Generally, the service lives of the detection module 100, the program module 101, and the infusion module 102 are different. Therefore, when the three are electrically connected to form a single device, the three can also be separated in pairs. For example, if one module expires, the user can only replace this module and keep the other two modules continuously using.

Here, it should be noted that the program module 101 of the embodiment of the present invention may also include multiple sub-modules. According to the functions of the sub-modules, different sub-modules can be respectively assembled in a different part, which is not a specific limitation herein, as long as the control conditions of the program module 101 can be satisfied.

Specifically, the program module 101 is preset with an rPID (risk-proportional-integral-derivative) algorithm that converts the asymmetric blood glucose in the original physical space to the approximately symmetric blood glucose in the risk space. The rPID algorithm is obtained by converting the classic PID (proportional-integral-derivative) algorithm. The specific converting method will be detailed below. According to the corresponding infusion instructions calculated by the rPID algorithm, module 101 controls the infusion Module 102 infuses insulin.

The classic PID algorithm can be expressed by the following formula:

$$PID ( t ) = C + {K}_{P} ( G - {G}_{B} ) + {K}_{I} \int ( G - {G}_{B} ) dt + {K}_{D} \frac{dG}{dt}$$

Where:
K_{P} is the gain coefficient of the proportional part;
K_{I} is the gain coefficient of the integral part;
K_{D} is the gain coefficient of the differential part;
G represents the current blood glucose level;
G_{B} represents the target blood glucose level;
C represents a constant;
PID (t) represents the infusion instruction sent to the insulin infusion system.

Considering the actual distribution characteristics of glucose concentration in diabetic patients, for example, the normal blood glucose range is 80-140 mg/dL, and it can also be widened to 70-180 mg/dL. General hypoglycemia can reach 20-40 mg/dL, while high blood glucose can reach 400-600 mg/dL.

The distribution of high/low blood glucose (original physical space) has significant asymmetry. In clinical practice, the risk of high blood glucose and low blood glucose corresponding to the same degree of blood glucose deviation from the normal range will be significantly different, such as a decrease of 70 mg/dL, from 120 mg/dL to 50 mg/dL will be considered severe hypoglycemia, with high clinical risk, and emergency measures such as supplementing carbohydrates need to be taken. The increase of 70 mg/dL, from 120 mg/dL to 190 mg/dL is just beyond the normal range. For diabetic patients, the degree of high blood glucose is not serious, and it is often reached in daily situations, and there is no need to take treatment measures.

Considering the asymmetric characteristics of the clinical risk of glucose concentration, the asymmetric blood glucose in the original physical space is converted to the approximately symmetric blood glucose in risk space, making the PID algorithm more robust.

Correspondingly, the rPID algorithm formula is converted into the following form:

$$rPID ( t ) = C + {K}_{P} r + {K}_{I} \int rdt + {K}_{D} \frac{dr}{dt}$$

Where:
rPID (t) represents the infusion instruction sent to the insulin infusion system after risk conversion;
r means blood glucose risk;
The meanings of other symbols are the same as described above.

In order to maintain the integration stability of PID, combined with the physiological effect of insulin to lower blood glucose, in one embodiment of the present invention, input parameter of the PID, blood glucose deviation amount Ge=G−GB is processed, such as segmented weighting (example: GB=110 mg/dL), as follows:

$$\{ \begin{matrix}r = Ge , if {G}_{B} < G \leq 180 mg / dL \\ r = Ge * 0.5 , if 180 < G \leq 300 mg / dL \\ r = Ge * 0.2 , if 300 < G \leq 400 mg / dL \\ r = ( 400 - {G}_{B} ) * 0.2 , if G > 400 mg / dL\end{matrix}$$

In another embodiment of the present invention, a blood glucose value greater than the target blood glucose G_{B} is converted by the relative value, as follows:

$$\{ \begin{matrix}r = G - {G}_{B} = Ge , if G \leq {G}_{B} \\ r = 100 * \frac{G - {G}_{B}}{G} = 100 * \frac{Ge}{G} , if G > {G}_{B}\end{matrix}$$

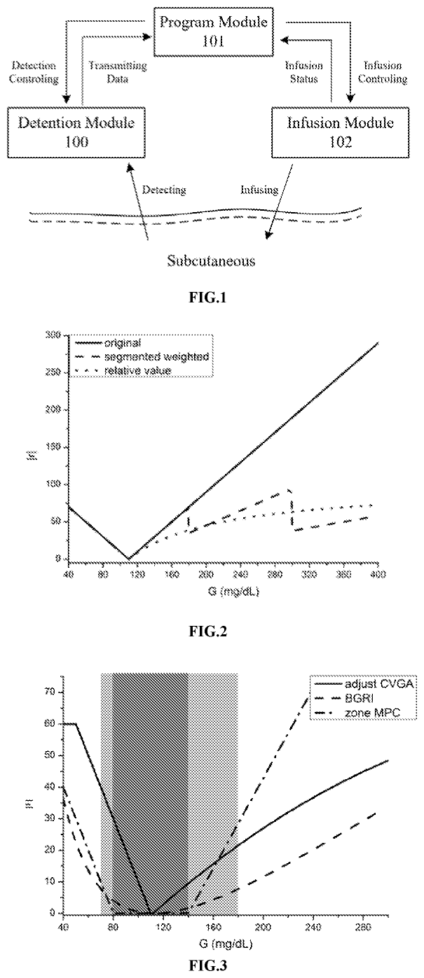

FIG. 2 is a comparison diagram of the blood glucose in the original physical space and the risk space obtained through the segmented weighting and the relative value conversion according to an embodiment of the present invention.

In the original PID algorithm, the blood glucose risk (ie Ge) on both sides of the target blood glucose value presents a severe asymmetry consisting of the original physical space. After being converted to the blood glucose risk space, the blood glucose risk on both sides of the target blood glucose value is approximately symmetric. In this way, the integral term can be kept stable, making the rPID algorithm more robust.

In another embodiment of the present invention, there is a fixed zero-risk point during risk conversion, and the data on both sides of the deviation from the zero-risk point is processed. The original parameter corresponding to greater than zero risk point is positive when converted to the risk space, and the original parameter corresponding to less than zero risk point is negative when converted to the risk space. Specifically, the classic blood glucose risk index (BGRI) method can be used. This method is based on clinical practice. It is believed that the clinical risks of 20 mg/dL for hypoglycemia and 600 mg/dL for hyperglycemia are equivalent. Through logarithm conversion, the overall blood glucose in the range of 20-600 mg/dL is processed. The blood glucose concentration at zero risk point in this method is set as G_{B}. The risk space conversion formula is as follows:

$$\{ \begin{matrix}r = - r ( G ) , if G \leq {G}_{B} ; \\ r = r ( G ) , if G > {G}_{B} ;\end{matrix}$$

where:

$$r ( G ) = 10 * {f ( G )}^{2}$$

The conversion function f(G) is as follows:

$$f ( G ) = 1.509 * [ {( ln ( G ) )}^{1.084} - 5.381 ]$$

In the classic blood glucose risk index (BGRI) method, the blood glucose concentration at zero risk point is 112 mg/dL. In other embodiments of the present invention, the blood glucose concentration at the zero-risk point can also be adjusted in conjunction with clinical practice risks and data trends; there is no specific limitation here. When fitting the risk space of the blood glucose concentration where the blood glucose concentration is greater than that at zero risk point, the specific fitting method is not specifically limited.

In another embodiment of the present invention, an improved Control Variability Grid Analysis (CVGA) method is used. The blood glucose concentration at zero risk point is defined as 110 mg/dL in the original CVGA, and the following equal-risk blood glucose concentration data pairs are assumed (90 mg/dL, 180 mg/dL; 70 mg/dL, 300 mg/dL: 50 mg/dL, 400 mg/dL). In the embodiment of the present invention, considering the real risks of clinical practice and the trend characteristics of the data, it was adjusted, and the risk data of (70 mg/dL, 300 mg/dL) was revised to (70 mg/dL, 250 mg/dL), and blood glucose concentration at zero risk point is defined as G_{B}. At the same time, a polynomial model is fitted to it, and the following risk functions for the two sides of the zero-risk point are obtained:

$$\{ \begin{matrix}r = G - {G}_{B} , if G \leq {G}_{B} ; \\ r = - 4.8265 * {10}^{4} - 4 * {G}^{2} + 0.45563 * G - 44.855 , if G > {G}_{B}\end{matrix}$$

And the maximum value is limited as:

$$\vert r \vert = min ( \vert r \vert , n )$$

Where the range of the limit of the maximum value n is from 0 to 80 mg/dL, preferably, the value of n is 60 mg/dL.

In other embodiments of the present invention, the blood glucose concentration at the zero-risk point and equal risk data pairs can also be adjusted in conjunction with clinical practice risks and data trends, and there is no specific limitation here. When fitting equal risk data pairs, the specific fitting method is not specifically limited. The data used to limit the maximum is also not specifically limited here.

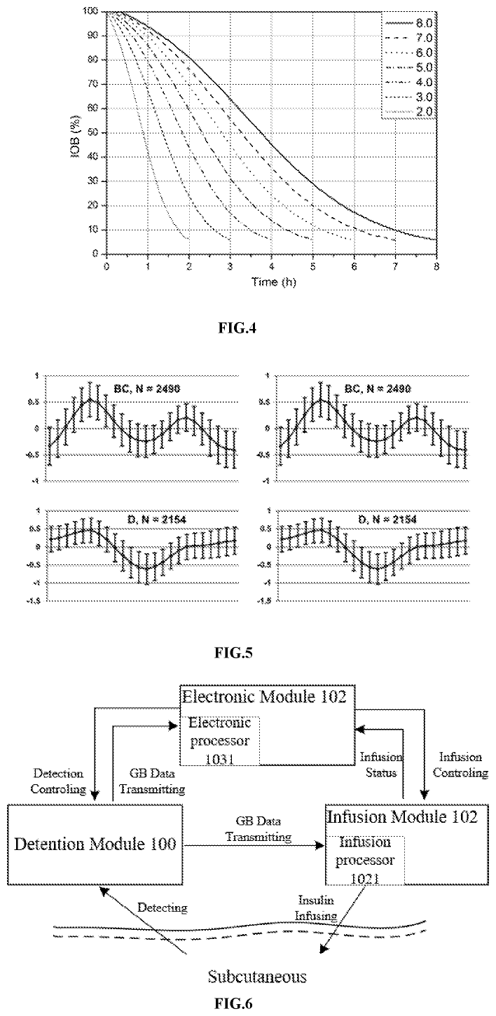

FIG. 3 is a comparison diagram of the blood glucose in the original physical space and the risk space, which has been obtained through the BGRI and CVGA method according to an embodiment of the present invention.

Similar to the treatment of Zone-MPC, within the normal range of blood glucose, the blood glucose risk after conversion by BGRI and CVGA methods is quite flat, especially within 80-140 mg/dL. Unlike Zone-MPC, where the blood glucose risk is completely zero in this range, it loses the ability to adjust further. Although the blood glucose risk in rPID is smooth within this range, it still has a stable and slow adjustment ability, making blood glucose further adjust to close the target value to achieve more precise blood glucose control.

In another embodiment of the present invention, a unified processing method can be used for data deviating from both sides of the zero-risk point. As in the preceding embodiment, the BGRI or CVGA method can deal with the data deviating from both sides of the zero-risk point: Different treatment methods can also be used, such as combining the BGRI and CVGA methods at the same time. The glucose concentration at zero risk point blood is the same, such as G_{B}. When the blood glucose concentration is less than G_{B}, the BGRI method is used, and the blood glucose concentration is greater than G_{B}, the CVGA method is used. At this time;

$$r = - r ( G ) , if G ⩽ {G}_{B}$$

where:

$$r ( G ) = 10 * {f ( G )}^{2}$$

The conversion function f(G) is as follows:

$$\begin{matrix}f ( G ) = 1.509 * [ {( ln ( G ) )}^{1.084} - 5.381 ] \\ r = - 4.8265 * {10}^{4} - 4 * {G}^{2} + 0.45563 * G - 44.855 , if G > {G}_{B}\end{matrix}$$

Similarly, when the blood glucose concentration is great than G_{B}, the BGRI method is used, and the blood glucose concentration is less than G_{B}, the CVGA method is used. At this time;

$$r = r ( G ) , if G > {G}_{B}$$

where:

$$r ( G ) = 10 * {f ( G )}^{2}$$

The conversion function f(G) is as follows:

$$f ( G ) = 1.509 * [ {( ln ( G ) )}^{1.084} - 5.3 \; 81 ]$$

$$r = G - {G}_{B} , if G > {G}_{B}$$

And the maximum value is limited as:

$$\vert r \vert = min ( \vert r \vert , n )$$

Where the range of the limit of the maximum value n is from 0 to 80 mg/dL, preferably, the value of n is 60 mg/dL.

In other embodiments of the present invention, the blood glucose level at the zero risk point can also be set as the target blood glucose value G_{B}, when the blood glucose concentration is less than G_{B}, the BGRI method is used, when the blood glucose concentration is great than G_{B}, such as segmented weighting or relative value converting.

When it is converted by segmented weighting, the formula is:

$$r = - r ( G ) , if G ⩽ {G}_{B}$$

where:

$$r ( G ) = 10 * {f ( G )}^{2}$$

The conversion function f(G) is as follows:

$$f ( G ) = 1.509 * [ {( ln ( G ) )}^{1.084} - 5.3 \; 81 ]$$

$$\{ \begin{matrix}r = Ge , if {G}_{B} < G \leq 180 mg / dL \\ r = Ge * 0.5 , if 180 < G \leq 300 mg / dL \\ r = Ge * 0.2 , if 300 < G \leq 400 mg / dL \\ r = ( 400 - {G}_{B} ) * 0.2 , if G > 400 mg / dL\end{matrix}$$

When it is converted by a relative value, the formula is:

$$r = - r ( G ) , if G ⩽ {G}_{B}$$

where:

$$r ( G ) = 10 * {f ( G )}^{2}$$

The conversion function f(G) is as follows:

$$f ( G ) = 1.509 * [ {( ln ( G ) )}^{1.084} - 5.3 \; 81 ]$$

$$r = 10 \; 0 * \frac{G - {G}_{B}}{G} = 100 * \frac{Ge}{G} , if G > {G}_{B}$$

When the blood glucose value at the zero risk point is the target blood glucose value G_{B}, for the data less than to the target blood glucose value G_{B}, when the segmented weighting converting, relative value converting, and CVGA method are used, the functions are the same. Therefore, when the blood glucose concentration is great than G_{B}, the BGRI method is used, when the blood glucose concentration is less than G_{B}, such as segmented weighting or relative value converting, the result is equivalent to the result that when the blood glucose value is less than the target blood glucose value G_{B}, the CVGA method is used when the blood glucose level is greater than the target blood glucose value G_{B}, the BGRI method is used, and the calculation formula is not repeated here.

In each embodiment of the present invention, the target blood glucose value G_{B} is 80-140 mg/dL; preferably, the target blood glucose value G_{B} is 110-120 mg/dL.

Through the above-converting methods, the asymmetric blood glucose in the original physical space can be converted to the approximately symmetric blood glucose in risk space in the rPID algorithm to retain the simplicity and robustness of the PID algorithm and control blood glucose risk with clinical value, to achieve precise control of the closed-loop artificial pancreatic insulin infusion system.

There are three major delay effects in the closed-loop artificial pancreas control system: insulin absorption delay (about 20 minutes from subcutaneous to blood circulation tissue, and about 100 minutes to liver), insulin onset delay (about 30-100 minutes), interstitial fluid glucose concentration and blood glucose detecting delay (approximately 5-15 minutes). Any attempt to accelerate the closed-loop responsiveness may result in unstable system behaviour and system oscillations. In order to compensate for the insulin absorption delay in the closed-loop artificial pancreas control system, in one embodiment of the present invention, an insulin feedback compensation mechanism is introduced. The amount of insulin that has not been absorbed in the body is subtracted from the output, which is a component that is proportional to the estimated plasma insulin concentration γ* [symbol] (t) (the plasma insulin concentration also regulates the actual human insulin secretion as a negative feedback Signal). The formula is as follows:

$${PID}_{c} ( t ) = PID ( t ) - γ * ( t )$$

### Where:

PID (t) represents the infusion instruction sent to the insulin infusion system;
PIDc(t) represents the infusion instruction with compensation sent to the insulin infusion system;
γ represents the compensation coefficient of the estimated plasma insulin concentration to the algorithm output. If the coefficient increases, the algorithm will be relatively conservative, and if the coefficient decreases, the algorithm will be relatively aggressive. Therefore, in the embodiment of the present invention, the range of γ is 0.4-0.6. Preferably, γ is 0.5.

[symbol] (t) represents the estimation of plasma insulin concentration, which various conventional prediction algorithms can obtain, for example, directly calculated from the infused insulin according to the pharmacokinetic curve of insulin, or using conventional autoregressive methods:

$$( n ) = {K}_{0} * {PID}_{c} ( n - 1 ) + {K}_{1} * ( n - 1 ) + {K}_{2} * ( n - 2 )$$

### Where:

[symbol] (n) represents the estimation of the plasma insulin concentration at the current moment;
PID_{c}(n−1) represents the output with compensation at the previous moment;

[symbol] (n−1) represents the estimation of the plasma insulin concentration at the previous moment;
[symbol] (n−2) represents the estimation of the plasma insulin concentration at the time of up and up;
K₀ represents the coefficient of the output part with compensation at the previous moment;
K₁ represents the coefficient of the estimated part of the plasma insulin concentration at the previous moment;
K₂ represents the coefficient of the estimated part of the plasma insulin concentration at the previous time;
Where: [symbol] (0)=K₀*PID_{c}(0), the time interval can be selected according to actual needs.

Correspondingly, the compensation output formula after risk conversion through the aforementioned method is as follows:

$${rPID}_{c} ( t ) = rPID ( t ) - γ * ( t )$$

### Where:

rPID_{c}(t) represents the infusion instruction with compensation sent to the insulin infusion system after risk conversion;

The meanings of the other characters are as described above.

In order to compensate for the delay of insulin onset in the closed-loop artificial pancreas control system, in one embodiment of the present invention, insulin on board (IOB), which has not yet worked in the body, is introduced, and the IOB is subtracted from the output of insulin to prevent accumulation and overdose for insulin infusion, which can lead to risks such as postprandial hypoglycemia.

FIG. 4 is an insulin IOB curve according to an embodiment of the present invention.

According to the IOB curve shown in FIG. 4, the cumulative residual amount of insulin previously infused can be calculated, and the selection of the specific curve can be determined based on the actual insulin action time of the user.

$${PID}^{′} ( t ) = PID ( t ) - IOB ( t )$$

### Where:

PID′ (t) represents the infusion instruction sent to the insulin infusion system after deducting IOB;
PID (t) represents the infusion instruction sent to the insulin infusion system;
IOB(t) represents the amount of insulin that has not yet worked in the body at time t.

Correspondingly, the output formula after deducting the amount of insulin that has not yet worked in the body after risk conversion through the aforementioned method is as follows:

$${rPID}^{′} ( t ) = rPID ( t ) - IOB ( t )$$

### Where:

rPID′ (t) represents the infusion instruction sent to the insulin infusion system after risk conversion, deducting the amount of insulin that has not yet worked in the body;

The meanings of the other characters are as described above.

In order to obtain an ideal control effect, IOB(t) is divided into meal insulin IOBm and non-meal insulin IOBo. The formula is as follows:

$$IOB ( t ) = {IOB}_{m , t} + {IOB}_{o , t}$$

### Where:

$$\{ \begin{matrix}{IOB}_{m , t} = {D}_{2} {I}_{m , t} \\ {IOB}_{o , t} = {D}_{2} {I}_{o , t} if G > 300 mg / dL \\ {IOB}_{o , t} = {D}_{4} {I}_{o , t} if 300 mg / dL \geq G > 200 mg / dL \\ {IOB}_{o , t} = {D}_{6} {I}_{o , t} if 200 mg / dL \geq G > 140 mg / dL \\ {IOB}_{o , t} = {D}_{8} {I}_{o , t} if G \leq 140 mg / dL\end{matrix}$$

### Where:

IOB_{m,t} represents the amount of meal insulin that has not yet worked in the body at time t;
IOB_{o,t} represents the amount of non-meal insulin that has not yet worked in the body at time t;
Di(i=2-8) represents the respective coefficients corresponding to the IOB curve with insulin action time i;
I_{m,t} represents the amount of meal insulin;
I_{o,t} represents the amount of non-meal insulin;
IOB(t) represents the amount of insulin that has not yet worked in the body at time t.

Dividing the IOB into meal and non-meal insulin can make insulin cleared faster when meals ingesting or blood sugar are too high and can obtain greater insulin output and regulate blood glucose more quickly. When approaching the target, a longer insulin action time curve is used to make insulin clear more slowly, and blood sugar regulation is more conservative and stable.

When PID′ (t)>0 or rPID′ (t)>0, the final insulin infusion amount is PID′ (t) or rPID′ (t);
When PID′ (t)<0 or rPID′ (t)<0, the final insulin infusion amount is 0.

In an embodiment of the present invention, an autoregressive method is used to compensate for detecting delay of interstitial fluid glucose concentration and blood glucose concentration. The formula is as follows:

$${G}_{S C} ( n ) = {K}_{0} * \hat{G} ( n - 1 ) + {K}_{1} * {G}_{S C} ( n - 1 ) + {K}_{2} * {G}_{S C} ( n - 2 )$$

### Where:

G_{SC}(n) represents the glucose concentration in the interstitial fluid at the current moment, that is, the measured value of the detecting system;
[symbol] (n−1) represents the estimated concentration of blood glucose at the previous moment;

G_{SC}(n−1) and G_{SC}(n−2) represent the glucose concentration in the interstitial fluid at the first previous time and the second previous time, respectively;
K₀ represents the coefficient of the estimated concentration of blood glucose at the previous moment;
K₀₁ and K₂ respectively represent the coefficient of glucose concentration in the interstitial fluid at the first previous time and the second previous time, respectively.

### Where: Ĝ (0)=G_{SC}(0)

The blood glucose concentration is estimated by the interstitial fluid glucose concentration, which compensates for the detecting delay of the interstitial fluid glucose concentration and blood glucose, making the PID algorithm more accurate. Correspondingly, the rPID algorithm can also more accurately calculate the actual insulin demand for the human body.

In the embodiment of the present invention, the insulin absorption delay, the insulin onset delay, the detecting delay of interstitial fluid glucose concentration and blood glucose can be partially compensated or fully compensated. Preferably, all delay factors are considered fully compensated for making the rPID algorithm more accurate.

In another embodiment of the present invention, the program module 101 is preset with an rMPC (risk-model-predict-control) algorithm that converts the asymmetric blood glucose in the original physical space to the approximately symmetric blood glucose in the risk space. The rMPC algorithm is obtained by converting the classic MPC (risk-model-predict-control) algorithm. According to the corresponding infusion instructions calculated by the rMPC algorithm, program module 101 controls infusion Module 102 infuses insulin.

The classic MPC algorithm consists of three elements, the prediction model, the value function and the constraints. The classic MPC prediction model is as follows:

$$\begin{matrix}{x}_{t + 1} = A {x}_{t} + {BI}_{t} \\ {G}_{t} = C {x}_{t}\end{matrix}$$

### Where:

$${x}_{t + 1} = [ \begin{matrix}{G}_{t + 1} \\ {G}_{t} \\ {G}_{t - 1}\end{matrix} ] ;$$

xₜ₊₁ represents the state parameter at the next moment,

$${x}_{t} = [ \begin{matrix}G \\ {G}_{t - 1} \\ {G}_{t - 2}\end{matrix} ] ;$$

xₜ represents the current state parameter,
Iₜ represents the amount of insulin infusion at the current moment;
Gₜ represents the blood glucose concentration at the current moment.

The parameter matrix is as follows:

$$\begin{matrix}A = [ \begin{matrix}{b}_{l} & {b}_{2} & {b}_{3} \\ 1 & 0 & 0 \\ 0 & 1 & 0\end{matrix} ] \\ B = [ \begin{matrix}{k}_{i} \\ 0 \\ 0\end{matrix} ] \\ C = [ 100 ]\end{matrix}$$

### Where:

b1, b2, b3, Ki are initial values.

The value function of the MPC algorithm is composed of the sum of squared deviations of the output G (blood glucose level) and the sum of squared changes of the input I (insulin amount). The MPC algorithm needs to obtain the minimum solution of the value function.

$${\int }^{MPC} = {\sum }_{j = 1}^{p} {‖ {G}_{t + j}^{e} ‖}^{2} + R {\sum }_{j = 1}^{N} {‖ {I}_{t + j}^{′} ‖}^{2}$$

### Where:

I′ₜ₊ⱼ represents the change of insulin infusion after step j;
Gₜ₊ⱼ^{e}, represents the difference between the predicted blood glucose concentration and the target blood glucose value after step j;
t represents the current moment;
N and P are the number of steps in the control time window and the predictive time window, respectively;
R is the weighting coefficient of the insulin component.
The amount of insulin infusion at step j is Iₜ+I′ₜ₊ⱼ.

In the embodiment of the present invention, the control time window Tc=30 min, the prediction time window Tp=60 min, and the weighting coefficient R of the amount of insulin is 11000. It should be noted that although the control time window used in the calculation is 30 min, only the first step calculation result of insulin output is used in the actual operation. After the operation, the minimum solution of the above value function is recalculated according to the latest blood glucose data obtained.

In the embodiment of the present invention, the infusion time step in the control time window is jₙ, and the range of jₙ is 0-30 min, preferably 2 min. The number of steps N=T/jₙ, and the range of j is 0 to N.

In other embodiments of the present invention, the weighting coefficients of the amount of insulin, the control time window and the predicted time window can also be selected as other values, which are not specifically limited here.

As mentioned above, the distribution of high/low blood glucose (original physical space) has significant asymmetry. The risk of high blood glucose and low blood glucose corresponding to the same degree of blood glucose deviation from the normal range will be significantly different in clinical practice. Considering the asymmetric characteristics of the clinical risk of glucose concentration, the asymmetric blood glucose in the original physical space is converted to the approximately symmetric blood glucose in risk space, making the MPC algorithm more accurate and flexible.

The value function of the rMPC algorithm after risk conversion is as follows:

$${\int }^{rMPC} = {\sum }_{j = 1}^{p} {‖ {r}_{t + j} ‖}^{2} + R {\sum }_{j = 1}^{N} {‖ {I}_{t + j}^{′} ‖}^{2}$$

### Where:

rₜ₊ⱼ represents the blood glucose risk index after step j;
I′ₜ₊ⱼ represents the change of insulin infusion after step j.

The deviation of blood glucose value is converted to the corresponding blood glucose risk. The specific conversion method is the same as that in the aforementioned rPID algorithm, such as segmented weighting and relative value converting; it also includes setting a fixed zero risk point in the risk space. The blood glucose concentration at the zero risk point can be set as the target blood glucose value. Data on both sides deviating from the zero risk point are processed, such as using BGRI and the improved CVGA method; it also includes different methods for processing data that deviates from the target blood glucose value.

Specifically, when the segmented weighting converting is used:

$$\{ \begin{matrix}{r}_{t + j} = ( {G}_{t + j} - {G}_{B} ) , if {G}_{B} < {G}_{t + j} \leq 180 mg / dL \\ {r}_{t + j} = ( {G}_{t + j} - {G}_{B} ) * 0.5 , if 180 < {G}_{t + j} \leq 300 mg / dL \\ {r}_{t + j} = ( {G}_{t + j} - {G}_{B} ) * 0.2 , if 300 < {G}_{t + j} \leq 400 mg / dL \\ {r}_{t + j} = ( 400 - {G}_{B} ) * 0.2 , if {G}_{t + j} > 400 mg / dL\end{matrix}$$

When the relative value converting is used:

$$\{ \begin{matrix}{r}_{t + j} = {G}_{t + j} - {G}_{B} , if {G}_{t + j} \leq {G}_{B} \\ {r}_{t + j} = 100 * \frac{{G}_{t + j} - {G}_{B}}{{G}_{t + j}} , if {G}_{t + j} > {G}_{B}\end{matrix}$$

When the BGRI method is used:

$$\{ \begin{matrix}{r}_{t + j} = - r ( {G}_{t + j} ) , if {G}_{t + j} \leq {G}_{B} \\ {r}_{t + j} = r ( {G}_{t + j} ) , if {G}_{t + j} > {G}_{B}\end{matrix}$$

### Where:

$$r ( {G}_{t + j} ) = 10 * {f ( {G}_{t + j} )}^{2}$$

The conversion function f(Gₜ₊ⱼ) is as follows:

$$f ( {G}_{t + j} ) = 1.509 * [ {( ln ( {G}_{t + j} ) )}^{1.084} - 5.381 ]$$

When the CVGA method is used:

$$\{ \begin{matrix}{r}_{t + j} = {G}_{t + j} - {G}_{B} , if {G}_{t + j} \leq {G}_{B} \\ {r}_{t + j} = - 4.8265 * 1 {0}^{4} - 4 * {G}_{t + j}^{2} + 0.45563 * {G}_{t + j} - 44.855 , if {G}_{t + j} > {G}_{B}\end{matrix}$$

And the maximum value is limited as:

$$\vert {r}_{t + j} \vert = min ( \vert {r}_{t + j} \vert , n )$$

Where the range of the limit of the maximum value n is from 0 to 80 mg/dL, preferably, the value of n is 60 mg/dL.

If the detected blood glucose concentration in step j Gₜ₊ⱼ is less than G_{B}, the BGRI method will be used. If the detected blood glucose concentration in step j Gₜ₊ⱼ is greater than G_{B}, the CVGA method will be used:

$${r}_{t + j} = - r ( {G}_{t + j} ) , if {G}_{t + j} \leq {G}_{B}$$

### Where:

$$r ( {G}_{t + j} ) = 10 * {f ( {G}_{t + j} )}^{2}$$

The conversion function f(Gₜ₊ⱼ) is as follows:

$$\begin{matrix}f ( {G}_{t + j} ) = 1.509 * [ {( ln ( {G}_{t + j} ) )}^{1.084} - 5.381 ] \\ {r}_{t + j} = - 4.8265 * 1 {0}^{4} - 4 * {G}_{t + j}^{2} + 0.45563 * {G}_{t + j} - 44.8 \; 55 , if {G}_{t + j} > {G}_{B}\end{matrix}$$

If the detected blood glucose concentration in step j Gₜ₊ⱼ is great than G_{B}, the BGRI method will be used. If the detected blood glucose concentration in step j Gₜ₊ⱼ is less than G_{B}, the CVGA method will be used:

$${r}_{t + j} = r ( {G}_{t + j} ) , if {G}_{t + j} > {G}_{B}$$

### Where:

$$r ( {G}_{t + j} ) = 10 * {f ( {G}_{t + j} )}^{2}$$

The conversion function f(Gₜ₊ⱼ) is as follows:

$$\begin{matrix}f ( {G}_{t + j} ) = 1.509 * [ {( ln ( {G}_{t + j} ) )}^{1.084} - 5.381 ] \\ {r}_{t + j} = {G}_{t + j} - {G}_{B} , if {G}_{t + j} \leq {G}_{B}\end{matrix}$$

And the maximum value is limited as:

$$\vert r \vert = min ( \vert r \vert , n )$$

Where the range of the limit of the maximum value n is from 0 to 80 mg/dL, preferably, the value of n is 60 mg/dL.

If the detected blood glucose concentration in step j Gₜ₊ⱼ is less than G_{B}, the BGRI method will be used. If the detected blood glucose concentration in step j Gₜ₊ⱼ is great than G_{B}, the segmented weighting converting will be used:

$${r}_{t + j} = - r ( {G}_{t + j} ) , if {G}_{t + j} \leq {G}_{B}$$

### Where:

$$r ( {G}_{t + j} ) = 10 * {f ( {G}_{t + j} )}^{2}$$

The conversion function f(Gₜ₊ⱼ) is as follows:

$$\begin{matrix}f ( {G}_{t + j} ) = 1.509 * [ {( ln ( {G}_{t + j} ) )}^{1.084} - 5.381 ] \\ \{ \begin{matrix}{r}_{t + j} = ( {G}_{t + j} - {G}_{B} ) , if {G}_{B} < {G}_{t + j} \leq 180 mg / dL \\ {r}_{t + j} = ( {G}_{t + j} - {G}_{B} ) * 0.5 , if 180 < {G}_{t + j} \leq 300 mg / dL \\ {r}_{t + j} = ( {G}_{t + j} - {G}_{B} ) * 0.2 , if 300 < {G}_{t + j} \leq 400 mg / dL \\ {r}_{t + j} = ( 400 - {G}_{B} ) * 0.2 , if {G}_{t + j} > 400 mg / dL\end{matrix}\end{matrix}$$

When the detected blood glucose concentration in step j Gₜ₊ⱼ is less than G_{B}, the BGRI method is used, when the detected blood glucose concentration in step j Gₜ₊ⱼ is great than G_{B}, the relative value converting is used:

$${r}_{t + j} = - r ( {G}_{t + j} ) , if {G}_{t + j} \leq {G}_{B}$$

### Where:

$$r ( {G}_{t + j} ) = 10 * {f ( {G}_{t + j} )}^{2}$$

The conversion function f(Gₜ₊ⱼ) is as follows:

$$\begin{matrix}f ( {G}_{t + j} ) = 1.509 * [ {( ln ( {G}_{t + j} ) )}^{1.084} - 5.381 ] \\ {r}_{t + j} = 100 * \frac{{G}_{t + j} - {G}_{B}}{{G}_{t + j}} , if {G}_{t + j} > {G}_{B}\end{matrix}$$

For the data less than the target blood glucose value G_{B}, the functions are the same when the segmented weighting converting, relative value converting, and CVGA method is used. Therefore, when the blood glucose concentration is great than G_{B}, the BGRI method is used, when the blood glucose concentration is less than G_{B}, such as segmented weighting or relative value converting, the result is equivalent to the result that when the blood glucose value is less than the target blood glucose value G_{B}, the CVGA method is used when the blood glucose level is greater than the target blood glucose value G_{B}, the BGRI method is used, and the calculation formula is not repeated here.

It should be noted that in the above conversion formulas:
rₜ₊ⱼ represents the blood glucose risk index at step j;
Gₜ₊ⱼ represents the blood glucose level detected in step j.

The target blood glucose value G_{B} is 80-140 mg/dL, preferably, the target blood glucose value G_{B} is 110-120 mg/dL.

The beneficial effects after risk conversion and the comparison of the relationship between blood glucose and blood glucose risk are consistent with the rPID algorithm and will not be repeated here.

Similarly, in order to compensate for the insulin absorption delay, the insulin feedback compensation mechanism can be used; in order to compensate for the delay of insulin onset, IOB can be used: in order to compensate for detecting delay of interstitial fluid glucose concentration and blood glucose concentration, the autoregressive method can be used. The specific compensation method is also consistent with the rPID algorithm, specifically:

For insulin absorption delay, the compensation formula is as follows:

$${rI}_{c ( t + j )} = {I}_{t + j} - γ {\hat{I}}_{P} ( t + j )$$

### Where:

Iₜ₊ⱼ represents the infusion instruction sent to the insulin infusion system after step j.
rI_{c(t+j)} represents the infusion instruction with compensation sent to the insulin infusion system after step j;
γ represents the compensation coefficient of the estimated plasma insulin concentration to the algorithm output. If the coefficient increases, the algorithm will be relatively conservative, and if the coefficient decreases, the algorithm will be relatively aggressive. Therefore, in the embodiment of the present invention, the range of γ is 0.4-0.6. Preferably, γ is 0.5.
[symbol] (t+j) represents the estimation of plasma insulin concentration after step j.

For the delay of insulin onset, the compensation formula is as follows:

$${rI}_{t + j}^{′} = {rI}_{t + j} - IOB ( t + j )$$

### Where:

rI′ₜ₊ⱼ represents the infusion instruction sent to the insulin infusion system after deducting IOB at step j after risk conversion;
rIₜ₊ⱼ represents the infusion instruction sent to the insulin infusion system at step j after risk conversion;
IOB(t+j) represents the amount of insulin that has not yet worked in the body at time t+j.

Similarly, IOB(t+j) can be divided into meal insulin and non-meal insulin. The formula is as follows:

$$IOB ( t + j ) = {IOB}_{m , t + j} + {IOB}_{o , t + j}$$

### Where:

$$\{ \begin{matrix}{IOB}_{m , t + j} = {D}_{2} {I}_{m , t + j} \\ \begin{matrix}{IOB}_{o , t + j} = {D}_{2} {I}_{o , t + j} & if {G}_{t + j} > 300 mg / dL\end{matrix} \\ \begin{matrix}{IOB}_{o , t + j} = {D}_{4} {I}_{o , t + j} & if 300 mg / dL \geq {G}_{t + j} > 200 mg / dL\end{matrix} \\ \begin{matrix}{IOB}_{o , t + j} = {D}_{6} {I}_{o , t + j} & if 200 mg / dL \geq {G}_{t + j} > 140 mg / dL\end{matrix} \\ \begin{matrix}{IOB}_{o , t + j} = {D}_{8} {I}_{o , t + j} & if {G}_{t + j} \leq 140 mg / dL\end{matrix}\end{matrix}$$

### Where:

IOB_{m,t+j} represents the amount of meal insulin that has not yet worked in the body at time t+j;
IOB_{o,t+j} represents the amount of non-meal insulin that has not yet worked in the body at time t+j;
Di(i=2-8) represents the respective coefficients corresponding to the IOB curve with insulin action time i;
I_{m,t+j} represents the amount of meal insulin at time t+j;
I_{o,t+j} represents the amount of non-meal insulin at time t+j;
IOB(t+j) represents the amount of insulin that has not yet worked in the body at time t+j.
When rI′ₜ₊ⱼ>0, the final insulin infusion amount is rI′ₜ₊ⱼ;
When rI′ₜ₊ⱼ<0, the final insulin infusion amount is 0.

The autoregressive method is used to detect the delay of interstitial fluid glucose concentration and blood glucose concentration.

the formula is as follows:

$${G}_{SC} ( t + j ) = {K}_{0} * \hat{G} ( t + j - 1 ) + {K}_{1} * {G}_{SC} ( t + j - 1 ) + {K}_{2} * {G}_{SC} ( t + j - 2 )$$

### Where:

G_{SC}(t+j) represents the glucose concentration in the interstitial fluid at the time t+j, that is, the measured value of the detecting system;
[symbol] (t+j−1) represents the estimated concentration of blood glucose at the time t+j−1;
G_{SC}(t+j−1) and G_{SC}(t+j−2) represent the glucose concentration in the interstitial fluid at the time t+j−1 and t+j−2, respectively;
K₀ represents the coefficient of the estimated concentration of blood glucose at the time t+j−1;
K₀₁ and K₂ respectively represent the coefficient of glucose concentration in the interstitial fluid at the time t+j−1 and t+j−2, respectively.

### Where: Ĝ (0)=G_{SC}(0)

The beneficial effects of various compensation methods are consistent with those in the rPID algorithm, which will not be repeated here.

In the rMPC algorithm, it is preferable to compensate for the delay of insulin onset and the detecting delay of interstitial fluid glucose concentration and blood glucose concentration.

In another embodiment of the present invention, the compound artificial pancreas algorithm is preset in program module 101. The compound artificial pancreas algorithm includes a first algorithm and a second algorithm. When the detection module 100 detects the current blood glucose level and sends the current blood glucose level to the program module 101, the first algorithm calculates the first insulin infusion amount I₁, the second algorithm calculates the second insulin infusion amount 12, the compound artificial pancreas algorithm optimises the first insulin infusion amount Ī and the second insulin infusion amount I₂ to obtain the final insulin infusion, and send the final insulin infusion amount I₃ to the infusion module 102, and the infusion module 102 performs insulin infusion according to the final infusion amount I₃.

The first and second algorithms are classic PID algorithms, the classic MPC algorithm, the rMPC algorithm, or the rPID algorithm. The rMPC algorithm or rPID algorithm is an algorithm that converts blood glucose that is asymmetric in the original physical space to a blood glucose risk that is approximately symmetric in the risk space. The conversion method of blood glucose risk in rMPC algorithm and rPID algorithm is as described above.

If I₁₌₁₂, then I₃=I₁=I₂;
If I₁≠I₂, then substitutes the average arithmetic value of I₁ and I₂ into the first and second algorithm to optimise the parameters, and then recalculate the current insulin infusion amount I₁ and I₂. If the data are not the same, repeat the above process until I₃=I₁=I₂, that is:
\textcircled{1} obtain the average value Ī of the first insulin infusion amount I₁ and the second insulin infusion amount I₂, and

$$\overline{I} = \frac{{I}_{1} + {I}_{2}}{2} ;$$

\textcircled{2} substitute the average value Ī into the first algorithm and the second algorithm to adjust the algorithm parameters;
\textcircled{3} recalculate the first insulin infusion amount I₁ and the second insulin infusion amount I₂ based on the current blood glucose level and the first algorithm and the second algorithm with adjusted the parameters;
\textcircled{4} calculate steps \textcircled{1}˜\textcircled{3} cyclically until I₁=I₂ and the final insulin infusion amount I₃=I₁=I₂.

At this time, when the first algorithm or the second algorithm is PID or rPID algorithm, the algorithm parameter is K_{P}, and K_{D}=T_{D}/K_{P}, T_{P} can be 60 min-90 min, K_{I}=T_{I}*K_{P}, T_{I} can be 150 min-450 min. When the first algorithm or the second algorithm is the MPC or rPMC algorithm, the algorithm parameter is K.

If I₁≠I₂, then the weighted value of I₁ and I₂ is substituted into the first and second algorithms to optimise the parameters and then recalculate the current insulin infusion amount I₁ and I₂. If the data are not the same, adjust the weighting coefficient to repeat the above process until I₃=I₁₌₁₂, that is:
\textcircled{1} obtain the weighted value I̅′̅ of the first insulin infusion amount I₁ and the second insulin infusion amount I₂, and I̅′̅=α*I₁+β+I₂, where α and β are the weighting coefficients of the first insulin infusion amount I₁ and the second insulin infusion amount I₂, respectively.
\textcircled{2} substitute the average value I̅′̅ into the first algorithm and the second algorithm to adjust the algorithm parameters;
\textcircled{3} recalculate the first insulin infusion amount I₁ and the second insulin infusion amount I₂ based on the current blood glucose level and the first algorithm and the second algorithm with adjusted the parameters;
\textcircled{4} calculate steps \textcircled{1}˜\textcircled{3} cyclically until I₁=I₂, and the final insulin infusion amount I₃=I₁=I₂.

Similarly, when the first algorithm or the second algorithm is PID or rPID algorithm, the algorithm parameter is K_{P}, and K_{D}=T_{D}/K_{P}, T_{P} can be 60 min-90 min, K_{I}=T_{I}*K_{P}, T_{I} can be 150 min-450 min. When the first algorithm or the second algorithm is the MPC or rPMC algorithm, the algorithm parameter is K.

In the embodiment of the present invention, α and β can be adjusted according to the first insulin infusion amount I₁ and the second insulin infusion amount I₂. When I₁≥I₂, α≤β; when I₁≥≤I₂, α≥β; preferably, α+β=1. In other embodiments of the present invention, α and β may also be other value ranges, which are not specifically limited here.

When the calculation results of the two are the same, that is, I₃=I₁=I₂, it can be considered that the amount of insulin infusion at the current moment can make the blood glucose level reach the ideal level. Through the processing mentioned above, the algorithms are mutually referenced. Preferably, the first algorithm and the second algorithm are the rMPC algorithm and the rPID algorithm, which are mutually referenced to improve the accuracy of the output further and make the result more feasible and reliable.

In another embodiment of the present invention, the program module 101 also provides a memory that stores the user's historical physical state, blood glucose level, insulin infusion, and other information. Statistical analysis can be performed based on the information in the memory to obtain the current statistical analysis result 14, when I₁+I₂, compare I₁, I₂ and I₄ to calculate the final insulin infusion amount I₃, the one that is closer to the statistical analysis result 14 is selected as a result of the compound artificial pancreas algorithm, that is the final insulin infusion amount I₃, and the program module 101 sends the final insulin infusion amount I₃ to the infusion module 102 to infuse:

$${I}_{3} = \{ \begin{matrix}{I}_{1} , & \vert {I}_{1} - {I}_{4} \vert \leq \vert {I}_{2} - {I}_{4} \vert \\ {I}_{2} , & \vert {I}_{1} - {I}_{4} \vert > \vert {I}_{2} - {I}_{4} \vert\end{matrix}$$

Through comparison with historical data, the reliability of insulin infusion is ensured, on the other hand.

In another embodiment of the present invention, when I₁ and I₂ are inconsistent, and the difference is large, the blood glucose risk space conversion method in the rMPC algorithm and/or rPID algorithm and/or the compensation method regarding the delay effect can also be changed to adjust and make them more closely, and then finally determine the output result of the compound artificial pancreas algorithm through the above arithmetic average, weighting processing, or comparison with the statistical analysis result.

In another embodiment of the present invention, the closed-loop artificial pancreas control system further includes a meal recognition module and/or a motion recognition module, used to identify whether the user is eating or exercising. Commonly used meal identification can be determined based on the rate of blood glucose change and compared with a specific threshold. The rate of blood glucose change can be calculated from two moments or obtained by linear regression at multiple moments within a period of time. Specifically, when the rate of change at the two moments is used for calculation, the calculation formula is:

$${dG}_{t} / dt = ( {G}_{t} - {G}_{t - 1} ) / Δ t$$

where:
Gₜ represents the blood glucose level at the current moment;
Gₜ₋₁ represents the blood glucose level at the previous moment;
Δt represents the time interval between the current moment and the last moment.
   When the rate of change at three moments is used for calculation, the calculation formula is:

$${dG}_{t} / dt = ( 3 {G}_{t} - 4 {G}_{t - 1} + {G}_{t - 2} ) / 2 Δ t$$

where:
Gₜ represents the blood glucose level at the current moment;
Gₜ₋₁ represents the blood glucose level at the previous moment;
Gₜ₋₂ represents the blood glucose level at the second previous moment
Δt represents the time interval between the current moment and the last moment.

Before calculating the blood glucose change rate, the original continuous glucose data can also be filtered or smoothed. The threshold can be set to 1.8 mg/mL-3 mg/mL or personalised.

Similar to meal recognition, exercise can cause a rapid drop in blood glucose. Therefore, exercise recognition can also be detected based on the rate of blood glucose change and a specific threshold. The rate of blood glucose change can also be calculated as described above, and the threshold can be personalised.

In order to determine the occurrence of movement more quickly, the closed-loop artificial pancreas insulin infusion control system further includes a movement sensor (not shown). The motion sensor automatically detects the user's physical activity, and the program module 101 can receive physical activity status information. The motion sensor can automatically and accurately sense the user's physical activity state and send the activity state parameters to the program module 101 to improve the output reliability of the compound artificial pancreas algorithm in exercise scenarios.

The motion sensor is provided in detection module 100, the program module 101 or the infusion module 102. Preferably, in the embodiment of the present invention, the motion sensor is provided in the program module 101.

It should be noted that the embodiment of the present invention does not limit the number of motion sensors and the installation positions of these multiple motion sensors, as long as the conditions for the motion sensor to sense the user's activity status can be satisfied.

The motion sensor includes a three-axis acceleration sensor or a gyroscope. The three-axis acceleration sensor or gyroscope can more accurately sense the body's activity intensity, activity mode or body posture. Preferably, in the embodiment of the present invention, the motion sensor combines a three-axis acceleration sensor and a gyroscope.

It should be noted that in the calculation process, the blood glucose risk conversion methods used by the rMPC algorithm and the rPID algorithm can be the same or different, and the compensation methods for the delay effect can also be the same or different. The calculation process can also be adjusted based on actual conditions.

In another embodiment of the present invention, the program module 101 provides an adaptive unit that adjusts the algorithm gain coefficient according to the user's weight. In some embodiments of the invention, the infusion module 102 or the program module 101 can indicate the user's daily insulin requirement DIR. In the embodiment of the invention, DIR can be calculated by body weight BW. Specifically, DIR is proportional to BW, that is, DIR=e*BW, where e is the weight adjustment coefficient.

For patients with type 1 diabetes, the weight adjustment coefficient e can be set as the population mean value, 0.53 U/kg, and it can also be customised according to their exercise habits. For example, a lower weight adjustment coefficient can be used for professional sports patients, such as 0.4 U/kg: for patients less involved in the exercise, a higher weight adjustment factor can be used, such as 0.6 U/kg. For patients with type 2 diabetes, a personalised weight adjustment factor can be selected in a larger range based on their pancreatic secretion function and insulin resistance, such as 0.1-1.5 U/kg, and the more commonly used range is 0.6-1.1 U/kg.

In an embodiment of the present invention, the algorithm preset in the program module 101 is a classic PID algorithm or rPID algorithm, and the gain coefficient of the proportional part K_{P}=DIR/(BW*m), m is the user weight compensation coefficient, and the value is 50˜ 500, preferably, m is 135.

The integral part gain coefficient Kr and the differential part gain coefficient K_{P} of the PID algorithm or rPID algorithm can be converted into coefficients related to K_{P}, such as K_{D}=T_{D}/K_{P}, T_{P} can be set as 60-90 min, K_{I}=T_{I}*K_{P}, T_{I} can be set as 150 min-450 min. Large T_{P} and T make the algorithms too radical, while little T_{D} and T_{I} make the algorithms too conservative. The different coefficients can be set during daytime and night. For example, a smaller time parameter can be selected at night.

In another embodiment of the present invention, the algorithm preset in the program module 101 is the classic MPC algorithm or rMPC algorithm, and its gain coefficient K is related to weight BW,

$$K = \frac{c * s}{e * BW}$$

### Where:

c is the safety factor;
s is the clinical experience coefficient;
e is the weight adjustment coefficient.

According to the risk of nighttime hypoglycemia, the safety factor c is set as 1.25-3: the clinical experience coefficient s can be 1500, 1700, 1800, 2000, 2200, 2500, etc., which can be adjusted according to the clinical results, and there is no specific limitation here. In a preferred embodiment of the present invention, the clinical experience coefficient s is 1700: the range of the weight adjustment coefficient e is described above.

In the foregoing two embodiments, the gain coefficient K_{P} of the PID algorithm or rPID algorithm and the gain coefficient K of the MPC algorithm or rMPC algorithm can also be adjusted by introducing the coefficient Sb (t) related to the basal insulin requirement, correspondingly:

$${K}_{P}^{′} = {K}_{P} * Sb ( t )$$

$${K}^{′} = K * Sb ( t )$$

The coefficient Sb (t) related to the basal insulin requirement is the ratio of the basal insulin requirement B (t) to the average of the daily basal insulin quantity Ba at time t, that is, Sb (t)=B (t)/Ba. Where, Ba=y*DIR/24, y is the basal insulin compensation coefficient, which takes a value of 0.1 to 5. The average population value of this coefficient is 0.47, and the data for children is slightly smaller, for example, 0.3-0.4.

The daily basal insulin quantity Ba average can be calculated according to the user's actual basal rate setting. The basal insulin requirement B (t) at time t can be set according to the four mainstream clinical optimal basal rate settings. FIG. 5 shows the four types of mainstream clinical optimal basal rate settings from the reference Holterhus, P M, J. Bokelmann, et al. (2013). “Predicting the Optimal Basal Insulin Infusion Pattern in Children and Adolescents on Insulin Pumps.” Diabetes Care 36 (6): 1507-1511, where the horizontal axis is time. 24 hours a day, and the vertical axis is the relative deviation between the basal insulin requirement and the average of the daily basal insulin quantity Ba at the corresponding time. Most of them are within [0.5, 1.5].

B (t) can also be set refer to the basic rate segmentation settings commonly used in clinical practice, such as three-stage settings, as follows:
\textcircled{1} When the time t is from 0) am to 4 am. B (t)=0.5DIR/48;
\textcircled{2} When the time t is 4 am to 10 am. B (t)=1.5DIR/48;
\textcircled{3} When the time t is from 10 am to 0 am. B (t)=DIR/48.

In other embodiments of the invention. B (t) can also be calculated according to the user-known and appropriate base rate setting.

In the embodiment of the present invention, the range of Sb (t) is 0.2-2, preferably 0.5-1.5. By introducing the coefficient Sb (t) related to the basal insulin requirement in different time periods, the gain coefficient is adjusted with the change of time to meet the user's insulin demand in different periods and further improve the accuracy of closed-loop control.

In other embodiments of the present invention, the conversion method of rPID algorithm and the rMPC algorithm, which converts the asymmetric blood glucose in the original physical space to the approximately symmetric blood glucose risk in risk space, and the processing method for the calculation result, and the beneficial effects are as described above, which will not be repeated here.

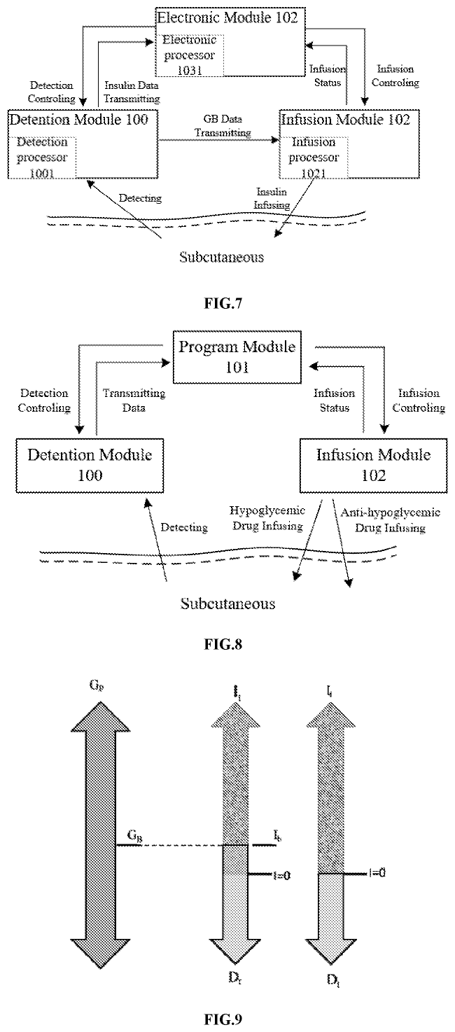

FIG. 6 is a schematic diagram of the module relationship of the closed-loop artificial pancreas insulin infusion control system according to another embodiment of the present invention.

In other embodiments of the present invention, the closed-loop artificial pancreas insulin infusion control system mainly includes a detection module 100, an infusion module 102, and an electronic module 103.

The detection module 100 is used to detect the user's real-time blood glucose level continuously. Generally, the detection module 100 is a continuous glucose monitor (Continuous Glucose Monitoring. CGM), which can detect blood glucose levels in real-time, monitor blood glucose changes, and send the current blood glucose levels to the infusion module 102 and the electronic module 103.

The infusion module 102 includes the mechanical assembly necessary for insulin infusion and other components capable of executing the first algorithm, such as an infusion processor 1021, controlled by the electronic module 103. The infusion module 102 receives the current blood glucose level sent by the detection module 100, calculates the first insulin infusion amount I₁ currently required through the first algorithm and sends the calculated first insulin infusion amount I₁ to the electronic module 103.

The electronic module 103 is used to control the operation of detection module 100 and the infusion module 102, Therefore, the electronic module 103 is connected to the detection module 100 and the infusion module 102, respectively, Here, the electronic module 103 is an external electronic device such as a mobile phone or a handset, and the connection refers to a wireless connection. The electronic module 103 includes a second processor. In the embodiment of the present invention, the second processor is capable of executing the second algorithm and the third algorithm, such as an electronic processor 1031, After the electronic module 103 receives the current blood sugar level, the current required second insulin infusion amount I₂ is calculated through the second algorithm. The first and second algorithms used by the electronic module 103 and the infusion module 102 to calculate the amount of insulin currently required are different.

After the electronic module 103 receives the first insulin infusion amount I₁ sent by the infusion module 102, it further optimises the first insulin infusion amount I₁ and the second insulin infusion amount I₂ through the third algorithm to obtain the final insulin infusion amount I₃, and sends final insulin infusion amount I₃ to the infusion module 102, the infusion module 102 injects the currently needed insulin amount I₃ into the user's body. At the same time, the infusion status of the infusion module 102 can also be fed back to the electronic module 103 in real-time. The specific optimisation method is as described above, which is:

If I₁=I₂, then I₃=I₁=I₂;

If I₁≠I₂, the electronic module 103 further substitutes the average arithmetic value of the two or the weighted value into the algorithm to recalculate the current insulin infusion amount I₁ and I₂. If the data are not the same, repeat the above process until I₃=I₁=I₂, that is:
\textcircled{1} obtain the average value Ī of the first insulin infusion amount I₁ and the second insulin infusion amount I₂, and

$$\overline{I} = \frac{{I}_{1} + {I}_{2}}{2} ;$$

\textcircled{2} substitute the average value Ī into the first algorithm and the second algorithm to adjust the algorithm parameters;
\textcircled{3} recalculate the first insulin infusion amount I₁ and the second insulin infusion amount I₂ based on the current blood glucose level and the first algorithm and the second algorithm with adjusted the parameters;
\textcircled{4} calculate steps \textcircled{1}˜\textcircled{3} cyclically until I₁=I₂, and the final insulin infusion amount I₃=I₁=I₂.

### Or:

\textcircled{1} obtain the average value I′ of the first insulin infusion amount I₁ and the second insulin infusion amount I₂, and I̅′̅=α*I₁+β*I₂, where α and β are the weighting coefficients of the first insulin infusion amount I₁ and the second insulin infusion amount I₂, respectively.
\textcircled{2} substitute the average value I′ into the first algorithm and the second algorithm to adjust the algorithm parameters;
\textcircled{3} recalculate the first insulin infusion amount I₁ and the second insulin infusion amount I₂ based on the current blood glucose level and the first algorithm and the second algorithm with adjusted the parameters;
\textcircled{4} calculate steps \textcircled{1}˜\textcircled{3} cyclically until I₁-I₂, and the final insulin infusion amount I₃=I₁=I₂.

When I₁≠I₂, the electronic module 103 can also compare I₁, I₂ and I₄, which is a statistical analysis result at the current time by analysing the historical information based on the user's body state, blood sugar level and insulin infusion at each time in the past. The one that is closer to the statistical analysis result 14 is selected as the final insulin infusion amount I₃, and the electronic module 103 sends the final insulin infusion amount I₃ to the infusion module 102 to infuse:

$${I}_{3} = \{ \begin{matrix}{I}_{1} , & \vert {I}_{1} - {I}_{4} \vert \leq \vert {I}_{2} - {I}_{4} \vert \\ {I}_{2} , & \vert {I}_{1} - {I}_{4} \vert > \vert {I}_{2} - {I}_{4} \vert\end{matrix}$$

In the embodiment of the present invention, the user's historical information may be stored in the electronic module 103 or a cloud management system (not shown), and the cloud management system and the electronic module 103 are connected wirelessly.

FIG. 7 is a schematic diagram of the module relationship of the closed-loop artificial pancreas insulin infusion control system according to another embodiment of the present invention.

In the embodiments of the present invention, the closed-loop artificial pancreas insulin infusion control system mainly includes a detection module 100, an infusion module 102, and an electronic module 103.

The detection module 100 is used to detect the user's real-time blood glucose level continuously. Generally, the detection module 100 is a continuous glucose monitor (Continuous Glucose Monitoring. CGM), which can detect blood glucose levels in real-time, monitor blood glucose changes, and the current blood glucose levels have only been sent to the infusion module 102. The detection module 100 further includes a second processor. In the embodiment of the present invention, the second processor is capable of executing the second algorithm, such as a detection processor 1001, After detecting the real-time blood glucose level, detection module 100 directly calculates the second insulin infusion amount I₂ through the second algorithm and sends the calculated second insulin infusion amount I₂ to the electronic module 103.

As mentioned above, infusion module 102, as mentioned above, after receiving the current blood glucose level sent by the detection module 100, calculates the first insulin infusion amount I₁ currently required through the first algorithm and sends the calculated first insulin infusion amount I₁ to the electronic module 103, The first and second algorithms used by the electronic module 103 and the infusion module 102 to calculate the amount of insulin currently required are different.

After the electronic module 103 receives the first insulin infusion amount I₁ sent by the infusion module 102 and the second insulin infusion amount I₂ sent by the detection module 103, it further optimises the first insulin infusion amount I₁ and the second insulin infusion amount I₂ through the third algorithm to obtain the final insulin infusion amount I₃. It sends the final insulin infusion amount I₃ to the infusion module 102. The infusion module 102 injects the currently needed insulin amount I₃ into the user's body. At the same time, the infusion status of the infusion module 102 can also be fed back to the electronic module 103 in real-time. The specific optimisation method is as described above.

In the above two embodiments of the present invention, after the detection module 100 detects the current blood glucose level, the infusion processor 1021 preliminarily calculates the first insulin infusion amount I₁. The second processor (such as the electronic processor 1031 and the detection processor 1001) preliminarily calculate the second insulin infusion amount 12, and I₁ and I₂ being sent to the electronic module 103. The electronic module 103 performs further optimisation and then sends the optimised final insulin infusion amount I₃ to the infusion module 102 to infuse insulin, improving the accuracy of infusion instructions.

In the above two embodiments of the present invention, the first algorithm and the second algorithm are one of the classic PID algorithms, the classic MPC algorithm, the rMPC algorithm, or the rPID algorithm. The advantages of using the rPID or rMPC algorithm to calculate are as described above, and the beneficial effects of other optimisation methods are also as described above and will not be repeated here.

The embodiment of the present invention does not limit the specific position and connection relationship of the detection module 100 and the infusion module 102, as long as the aforementioned functional conditions can be met.

As in an embodiment of the present invention, the two modules are electrically connected to form an integral assembly and are pasted in the same place on the user's skin. If the two modules are connected as a whole and pasted in the same position, the number of user skin pasting devices will be reduced, thereby reducing the interference of more pasted devices on user activities: at the same time, it also effectively solves the problem of poor wireless communication between separate devices, which further enhance the user experience.

As in another embodiment of the present invention, the two modules are arranged in different components and are passed on different positions of the user's skin. The detection module 100 and the infusion module 102 transmit wireless signals to realise the mutual connection.

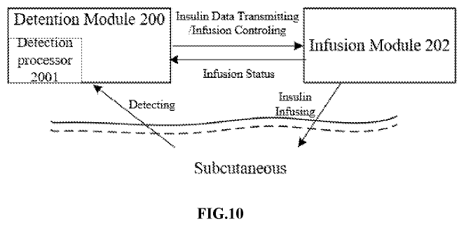

FIG. 8 is a schematic diagram of the module relationship of the closed-loop artificial pancreas multi-drug infusion control system according to another embodiment of the present invention.

In the embodiment of the present invention, the closed-loop artificial pancreas insulin infusion control system mainly includes a detection module 100, a program module 101, and an infusion module 102, The infusion module 102 can perform multi-drug infusion, and the drugs can be a combination for regulating blood glucose for diabetic patients, Its metabolite is glucose, the main drugs are hypoglycemic drugs, such as insulin and its analogue, and other combination drugs are anti-hypoglycemic drugs, which has opposite effects with hypoglycemic drugs, such as pancreatic hypertension Glucagon and its analogs, cortisol and its analogs, growth hormone and its analogs, epinephrine and its analogs, glucose, etc., dextrins with similar effects Analogs (such as pramlintide), etc.

The infusion module 102 can infuse the hypoglycemic drug and/or the anti-hypoglycemic drug into the user according to the hypoglycemic drug infusion instruction and/or the anti-hypoglycemic drug infusion instruction issued by the program module 101. The hypoglycemic and blood sugar raising drugs can be infused separately through different drug paths or through the same drug path at different times. The specific drug path design is not limited here.

FIG. 9 is a schematic diagram of dual-drug infusion switching according to two embodiments of the present invention.

In an embodiment of the present invention, the hypoglycemic drug infusion instruction and/or the current anti-hypoglycemic drug infusion instruction are obtained by comparing the predicted blood glucose concentration estimated G_{P} with the target blood glucose value G_{B}, and the predicted blood glucose concentration G_{P} may be predicted based on the prediction model of rMPC or other suitable blood glucose prediction algorithms: the hypoglycemic drug infusion data and/or the anti-hypoglycemic drug infusion data can be calculated by the aforementioned rMPC algorithm or rPID algorithm or compound artificial pancreas algorithm. Specifically:
When G_{P}>G_{B}, the infusion module 102 starts to infuse the hypoglycemic drug according to the hypoglycemic drug infusion data Iₜ, which is calculated by the rMPC algorithm or the rPID algorithm or the compound artificial pancreas algorithm;
When G_{P}<G_{B}, the infusion module 102 starts to infuse the anti-hypoglycemic drug infusion according to the anti-hypoglycemic drug infusion data Dr, which is calculated by the rMPC algorithm or the rPID algorithm or the compound artificial pancreas algorithm;

In the embodiment of the present invention. It represents the amount of hypoglycemic drugs that need to be infused to control blood glucose at the target blood glucose level G_{B} without interference. When G_{P}=G_{B}. Iₜ=I_{b}, when G_{P}>G_{B}, with the infusion of hypoglycemic drugs. G_{P} further decreases, and It also decreases. When the infusion module 102 has only one set of drug infusion paths, when G_{P}<G_{B}, that is, Iₜ<I_{b}, the infusion module 102 starts to infuse anti-hypoglycemic drugs, and the anti-hypoglycemic drug infusion data D, can be calculated by the rMPC algorithm or the rPID algorithm or the compound artificial pancreas algorithm, and the infusion of hypoglycemic drugs is stopped at the same time to prevent the hypoglycemic drugs and the anti-hyperglycemic drugs from affecting each other due to their antagonistic effects. When the infusion module 102 has at least two sets of drug infusion paths when 0≤Iₜ<I_{b}, the hypoglycemic drugs and anti-hyperglycemic can be infused simultaneously, which can effectively prevent hypoglycemia. When Iₜ<0, the infusion of hyperglycemic drugs is stopped and only infuse anti-hyperglycemic drugs.

In another embodiment of the present invention, the hypoglycemic drug infusion instruction and/or the current anti-hypoglycemic drug infusion instruction may be directly performed by comparing the required amount of the hypoglycemic drug Iₜ with the target hypoglycemic drug amount I_{b}, and the hypoglycemic drug required amount Iₜ and the target hypoglycemic drug amount I_{b} can be calculated by the aforementioned rMPC algorithm, rPID algorithm, or compound artificial pancreas algorithm, Specifically: when the infusion module 102 has at least two sets of drug infusion paths;

When Iₜ≥I_{b}, the infusion module 102 starts to infuse the hypoglycemic drug according to the hypoglycemic drug infusion data Iₜ, which is calculated by the rMPC algorithm or the rPID algorithm or the compound artificial pancreas algorithm;

When 0≤Iₜ<I_{b}, the hypoglycemic drugs and anti-hypoglycemic can be infused at the same time, which can effectively prevent the occurrence of hypoglycemia. The hypoglycemic drug required amount Iₜ and the target hypoglycemic drug amount I_{b} can be calculated by the aforementioned rMPC algorithm, rPID algorithm, or compound artificial pancreas algorithm.

When Iₜ<0, the infusion of hyperglycemic drugs is stopped and only infuse anti-hyperglycemic drugs. The anti-hypoglycemic drug infusion data D, can be calculated by the rMPC algorithm, rPID, compound artificial pancreas algorithm.

Preferably, in the embodiment of the present invention, the hypoglycemic is insulin, and the anti-hypoglycemic is glucagon.

It should be noted that in the above embodiments, the calculation methods of the hypoglycemic drug infusion data and the anti-hypoglycemic infusion data at each stage may be the same or different. Preferably, the same algorithm architecture ensures the basic conditions' consistency, which makes the calculation results more accurate. More preferably, the compound artificial pancreas algorithm is used for calculation, and the advantages of the rPID algorithm and the rMPC algorithm are fully utilised to face complex scenarios to make the blood glucose control ideally.

FIG. 10 is a schematic diagram of the module relationship of the closed-loop artificial pancreas insulin infusion control system according to another embodiment of the present invention.

The closed-loop artificial pancreas insulin infusion control system disclosed in the embodiment of the present invention mainly includes a detection module 200 and an infusion module 202. The detection module 100 is used to continuously detect the user's current blood glucose (BG) level. Generally, detection module 100 is a Continuous Glucose Monitoring (CGM) for detecting real-time BG and monitoring BG changes. The detection module 200 also includes a detection processing unit 2001. The detection processing unit 2001 is preset with an algorithm for calculating insulin amount for infusion. When the user's current blood glucose level is detected by the detection module 200, the detection processing unit 2001 calculates the insulin amount required by the user through the preset algorithm. The insulin amount required by the user is sent to infusion module 202.

The infusion module 202 includes the essential mechanical assemblies for insulin infusion and an electronic transceiver that receives the user's insulin amount information from the detection module 200. According to the current insulin infusion amount sent by the detection module 200, infusion module 202 infuses the currently required insulin into the user's body. At the same time, the infusion status of infusion module 202 can also be fed back to detection module 200 in real-time.

In the embodiment of the present invention, the algorithm for calculating the insulin infusion amount, preset in the detection processing unit 2001, is one of the classic PID algorithms, the classic MPC algorithm, the rMPC rPID algorithm or the compound artificial pancreas algorithm. The calculation method and beneficial effects of using rPID algorithm, rMPC. The algorithm or the compound artificial pancreas algorithm is described above and will not be repeated here.

The embodiment of the present invention does not limit the specific position and connection relationship of the detection module 200 and the infusion module 202, as long as the aforementioned functional conditions can be met.

As in an embodiment of the present invention, the two are electrically connected to form an integral assembly and are pasted in the same place on the user's skin. If the two modules are connected as a whole and pasted in the same position, the number of user skin pasting devices will be reduced, thereby reducing the interference of more pasted devices on user activities: at the same time, it also effectively solves the problem of poor wireless communication between separate devices, which further enhance the user experience.

As in another embodiment of the present invention, the two modules are arranged in different components and are passed on different positions of the user's skin. The detection module 100 and the infusion module 102 transmit wireless signals to realize the mutual connection.

In summary; the present invention discloses a closed-loop artificial pancreas insulin infusion control system, the system is preset with a rMPC algorithm, which converts the asymmetric blood glucose in the original physical space to the approximately symmetric blood glucose risk in the risk space, the recent changes of blood glucose and the asymmetry of distribution are taken into account in the rMPC algorithm, giving the rMPC algorithm the advantages of precision and flexibility, realizing precise control for closed-loop artificial pancreas insulin infusion system.

While the invention has been described in detail with reference to the specific embodiments of the present invention, it should be understood that it will be appreciated by those skilled in the art that the above embodiments may be modified without departing from the scope and spirit of the invention. The scope of the invention is defined by the appended claims.

1. A closed-loop artificial pancreas insulin infusion control system, comprising:
a detection module, configured to detect a current blood glucose level G continuously;
a program module, configured to preset with an rMPC algorithm that converts an asymmetric blood glucose in an original physical space to an approximately symmetric blood glucose risk in a risk space and a target blood glucose level G_{B}, and to calculate insulin infusion instructions based on a blood glucose risk by the rMPC algorithm; and
an infusion module, connected to the program module, and configured to infuse insulin, controlled by the program module, according to corresponding output instructions calculated by the rMPC algorithm.

2. According to the closed-loop artificial pancreas insulin infusion control system of claim 1, wherein,
the rMPC algorithm comprises a prediction model, a value function and constraints, where the prediction model is:
$${x}_{t + 1} = {Ax}_{t} + {BI}_{t}$$
$${G}_{t} = {Cx}_{t}$$
where:
xₜ₊₁ represents a state parameter at a next moment
$${x}_{t + 1} = [ \begin{matrix}{G}_{t + 1} \\ {G}_{t} \\ {G}_{t - 1}\end{matrix} ] ;$$
xₜ represents a current state parameter,
$${x}_{t} = [ \begin{matrix}{G}_{t} \\ {G}_{t - 1} \\ {G}_{t - 2}\end{matrix} ] ;$$
Iₜ represents an amount of insulin infusion at a current moment;
Gₜ represents a blood glucose concentration at the current moment;
a parameter matrix in the prediction model of the rMPC algorithm is as follows:
$$A = [ \begin{matrix}{b}_{1} & {b}_{2} & {b}_{3} \\ 1 & 0 & 0 \\ 0 & 1 & 0\end{matrix} ]$$
$$B = [ \begin{matrix}{k}_{i} \\ 0 \\ 0\end{matrix} ]$$
$$C = [ \begin{matrix}1 & 0 & 0\end{matrix} ]$$
where:
b1, b2, b3, Ki are prior values,
the value function is:
$${J}^{rMPC} = {\sum }_{j = 1}^{p} {‖ {r}_{t + j} ‖}^{2} + R {\sum }_{j = 1}^{N} {‖ {I}_{t + j}^{′} ‖}^{2}$$
where:
rₜ₊ⱼ represents a blood glucose risk index after step j;
I′ₜ₊ⱼ represents a change of insulin infusion after step j;
t represents the current moment;
N and P are the number of steps in a control time window and a predictive time window respectively;
R is a weighting coefficient of a insulin component.

3. According to the closed-loop artificial pancreas insulin infusion control system of claim 2, wherein,
the blood glucose risk index rₜ₊ⱼ is calculated as:
$$\{ \begin{matrix}{r}_{t + j} = ( {G}_{t + j} - {G}_{B} ) , & if {G}_{B} < {G}_{t + j} \leq 180 mg / dL \\ {r}_{t + j} = ( {G}_{t + j} - {G}_{B} ) * 0.5 , & if 180 < {G}_{t + j} \leq 300 mg / dL \\ {r}_{t + j} = ( {G}_{t + j} - {G}_{B} ) * 0.2 , & if 300 < {G}_{t + j} \leq 400 mg / dL \\ {r}_{t + j} = ( 400 - {G}_{B} ) * 0.2 , & if {G}_{t + j} > 400 mg / dL\end{matrix}$$
where:
Gₜ₊ⱼ represents a blood glucose level detected in step j.

4. According to the closed-loop artificial pancreas insulin infusion control system of claim 2, wherein,
the blood glucose risk index rₜ₊ⱼ is calculated as:
$$\{ \begin{matrix}{r}_{t + j} = {G}_{t + j} - {G}_{B} , & if {G}_{t + j} \leq {G}_{B} \\ {r}_{t + j} = 100 * \frac{{G}_{t + j} - {G}_{B}}{{G}_{t + j}} , & if {G}_{t + j} > {G}_{B}\end{matrix}$$
where:
Gₜ₊ⱼ represents a blood glucose level detected in step j.

5. According to the closed-loop artificial pancreas insulin infusion control system of claim 2, wherein,
the blood glucose risk index rₜ₊ⱼ is calculated as:
$$\{ \begin{matrix}{r}_{t + j} = - r ( {G}_{t + j} ) , & if {G}_{t + j} \leq {G}_{B} \\ {r}_{t + j} = r ( {G}_{t + j} ) , & if {G}_{t + j} > {G}_{B}\end{matrix}$$
where:
$$r ( {G}_{t + j} ) = 10 * {f ( {G}_{t + j} )}^{2}$$
a conversion function f(Gₜ₊ⱼ) is as follows:
$$f ( {G}_{t + j} ) = 1.509 * [ {( ln ( {G}_{t + j} ) )}^{1.084} - 5.381 ]$$
where:
Gₜ₊ⱼ represents a blood glucose level detected in step j.

6. According to the closed-loop artificial pancreas insulin infusion control system of claim 2, wherein,
the blood glucose risk index rₜ₊ⱼ is calculated as:
$$\{ \begin{matrix}{r}_{t + j} = {G}_{t + j} - {G}_{B} , & if {G}_{t + j} \leq {G}_{B} \\ \begin{matrix}{r}_{t + j} = - 4.8265 * {10}^{4} - 4 * {{G}_{t + j}}^{2} + \\ 0.45563 * {G}_{t + j} - 44.855 ,\end{matrix} & if {G}_{t + j} > {G}_{B}\end{matrix}$$
where:
Gₜ₊ⱼ represents a blood glucose level detected in step j.

7. According to the closed-loop artificial pancreas insulin infusion control system of claim 6, wherein,
a maximum value of the blood glucose risk index rₜ₊ⱼ is limited as:
$$\vert {r}_{t + j} \vert = min ( \vert {r}_{t + j} \vert , n ) .$$

8. (canceled)

9. (canceled)

10. According to the closed-loop artificial pancreas insulin infusion control system of claim 2, wherein,
when the detected blood glucose concentration in step j Gₜ₊ⱼ is less than the target blood glucose level G_{B}, a BGRI method is used, the blood glucose risk index rₜ₊ⱼ is calculated as:
$${r}_{t + j} = r ( {G}_{t + j} ) , if {G}_{t + j} > {G}_{B}$$
where:
$$r ( {G}_{t + j} ) = 10 * {f ( {G}_{t + j} )}^{2}$$
a conversion function f(Gₜ₊ⱼ) is as follows:
$$f ( {G}_{t + j} ) = 1.509 * [ {( ln ( {G}_{t + j} ) )}^{1.084} - 5.381 ]$$
when the detected blood glucose concentration in step j Gₜ₊ⱼ is greater than the target blood glucose level G_{B}, a CVGA method is used, the blood glucose risk index rₜ₊ⱼ is calculated as:
$${r}_{t + j} = {G}_{t + j} - {G}_{B} , if {G}_{t + j} \leq {G}_{B}$$
where:
Gₜ₊ⱼ represents the blood glucose level detected in step j.

11. According to the closed-loop artificial pancreas insulin infusion control system of claim 10, wherein,
a maximum value of the blood glucose risk index rₜ₊ⱼ is limited as:
$$\vert {r}_{t + j} \vert = min ( \vert {r}_{t + j} \vert , n ) .$$

12. (canceled)

13. (canceled)

14. According to the closed-loop artificial pancreas insulin infusion control system of claim 2, wherein,
when the detected blood glucose concentration in step j Gₜ₊ⱼ is less than the target blood glucose level G_{B}, a BGRI method is used, the blood glucose risk index rₜ₊ⱼ is calculated as:
$${r}_{t + j} = - r ( {G}_{t + j} ) , if {G}_{t + j} \leq {G}_{B}$$
where:
$$r ( {G}_{t + j} ) = 10 * {f ( {G}_{t + j} )}^{2}$$
a conversion function f(Gₜ₊ⱼ) is as follows:
$$f ( {G}_{t + j} ) = 1.509 * [ {( ln ( {G}_{t + j} ) )}^{1.084} - 5.381 ]$$
when the detected blood glucose concentration in step j Gₜ₊ⱼ is great than the target blood glucose level G_{B}, a segmented weighting converting is used, the blood glucose risk index rₜ₊ⱼ is calculated as:
$${r}_{t + j} = - 4.8265 * {10}^{4} - 4 * {{G}_{t + j}}^{2} + 0.45563 * {G}_{t + j} - 44.855 , if {G}_{t + j} > {G}_{B}$$
where:
Gₜ₊ⱼ represents the blood glucose level detected in step j.

15. According to the closed-loop artificial pancreas insulin infusion control system of claim 2, wherein,
when the detected blood glucose concentration in step j Gₜ₊ⱼ is less than the target blood glucose level G_{B}, a BGRI method is used, the blood glucose risk index rₜ₊ⱼ is calculated as:
$${r}_{t + j} = - r ( {G}_{t + j} ) , if {G}_{t + j} \leq {G}_{B}$$
where:
$$r ( {G}_{t + j} ) = 10 * {f ( {G}_{t + j} )}^{2}$$
a conversion function f(Gₜ₊ⱼ) is as follows:
$$f ( {G}_{t + j} ) = 1.509 * [ {( ln ( {G}_{t + j} ) )}^{1.084} - 5.381 ]$$
when the detected blood glucose concentration in step j Gₜ₊ⱼ is great than the target blood glucose level G_{B}, a segmented weighting converting is used, the blood glucose risk index rₜ₊ⱼ is calculated as:
$$\{ \begin{matrix}{r}_{t + j} = ( {G}_{t + j} - {G}_{B} ) , & if {G}_{B} < {G}_{t + j} \leq 180 mg / dL \\ {r}_{t + j} = ( {G}_{t + j} - {G}_{B} ) * 0.5 , & if 180 < {G}_{t + j} \leq 300 mg / dL \\ {r}_{t + j} = ( {G}_{t + j} - {G}_{B} ) * 0.2 , & if 300 < {G}_{t + j} \leq 400 mg / dL \\ {r}_{t + j} = ( 400 - {G}_{B} ) * 0.2 , & if {G}_{t + j} \geq 400 mg / dL\end{matrix}$$
where:
Gₜ₊ⱼ represents the blood glucose level detected in step j.

16. According to the closed-loop artificial pancreas insulin infusion control system of claim 2, wherein,
when the detected blood glucose concentration in step j Gₜ₊ⱼ is less than the target blood glucose level G_{B}, a BGRI method is used, the blood glucose risk index rₜ₊ⱼ is calculated as:
$${r}_{t + j} = - r ( {G}_{t + j} ) , if {G}_{t + j} \leq {G}_{B}$$
where:
$$r ( {G}_{t + j} ) = 10 * {f ( {G}_{t + j} )}^{2}$$
a conversion function f(Gₜ₊ⱼ) is as follows:
$$f ( {G}_{t + j} ) = 1.509 * [ {( ln ( {G}_{t + j} ) )}^{1.084} - 5.381 ]$$
when the detected blood glucose concentration in step j Gₜ₊ⱼ is great than the target blood glucose level G_{B}, a segmented weighting converting is used, the blood glucose risk index rₜ₊ⱼ is calculated as:
$${r}_{t + j} = 100 * \frac{{G}_{t + j} - {G}_{B}}{{G}_{t + j}} , if {G}_{t + j} > {G}_{B}$$
where:
Gₜ₊ⱼ represents the blood glucose level detected in step j.

17. (canceled)

18. (canceled)

19. According to the closed-loop artificial pancreas insulin infusion control system of claim 1, wherein,
the rMPC algorithm comprises one or more of the following processing methods:
\textcircled{1} according to an insulin absorption delay in the artificial pancreas control system, an amount of plasma insulin that is not absorbed in a body γ [symbol] (t+j) is deducted;
$${rI}_{c ( t + j )} = {I}_{t + j} - γ ( t + j )$$
where:
Iₜ₊ⱼ represents the infusion instruction sent to the insulin infusion control system after step j;
rI_{c(t+j)} represents the infusion instruction with compensation sent to the insulin infusion control system after the step j;
γ represents a compensation coefficient of an estimated plasma insulin concentration to an output of the rMPC algorithm; [symbol]
(t+j) represents an estimation of plasma insulin concentration in the step j;
\textcircled{2} according to an insulin onset delay in the artificial pancreas control system, an amount of insulin that has not yet worked in the body IOB(t+j) is deducted:
$${rI}_{t + j}^{′} = {rI}_{t + j} - IOB ( t + j )$$
where:
rI′ₜ₊ⱼ represents the infusion instruction sent to the insulin infusion control system after deducting IOB at the step j after risk conversion;
rIₜ₊ⱼ represents the infusion instruction sent to the insulin infusion control system at step the j after risk conversion;
IOB(t+j) represents the amount of insulin that has not yet worked in the body at time t+j;
\textcircled{3} an autoregressive method is used to compensate for a detecting delay of blood glucose concentration and an interstitial fluid glucose concentration.

20. According to the closed-loop artificial pancreas insulin infusion control system of claim 19, wherein,
the estimation of plasma insulin concentration [symbol] (t+j) in the step j is obtained by the autoregressive method.

21. (canceled)

22. (canceled)

23. According to the closed-loop artificial pancreas insulin infusion control system of claim 19, wherein,
the amount of insulin that has not yet worked in the body at the time t+j IOB(t+j) is obtained from IOB curves.

24. According to the closed-loop artificial pancreas insulin infusion control system of claim 19, wherein,
the amount of insulin that has not yet worked in the body at the time t+j IOB(t+j) is divided in to a meal insulin and a non-meal insulin;
$$IOB ( t + j ) = {IOB}_{m , t + j} + {IOB}_{o , t + j}$$
where:
$$\{ \begin{matrix}{IOB}_{m , t + j} = {D}_{2} {I}_{m , t + j} \\ \begin{matrix}{IOB}_{o , t + j} = {D}_{2} {I}_{o , t + j} & if {G}_{t + j} > 300 mg / dL\end{matrix} \\ \begin{matrix}{IOB}_{o , t + j} = {D}_{4} {I}_{o , t + j} & if 300 mg / dL \geq {G}_{t + j} > 200 mg / dL\end{matrix} \\ \begin{matrix}{IOB}_{o , t + j} = {D}_{6} {I}_{o , t + j} & if 200 mg / dL \geq {G}_{t + j} > 140 mg / dL\end{matrix} \\ \begin{matrix}{IOB}_{o , t + j} = {D}_{8} {I}_{o , t + j} & if {G}_{t + j} \leq 140 mg / dL\end{matrix}\end{matrix}$$
where:
IOB_{m,t+j} represents an amount of meal insulin that has not yet worked in the body at the time t+j;
IOB_{o,t}+j represents an amount of non-meal insulin that has not yet worked in the body at the time t+j;
Di(i=2-8) represents respective coefficients corresponding to the IOB curve with insulin action time i;
I_{m,t+j} represents the amount of meal insulin at the time t+j;
I_{o,t+j} represents the amount of non-meal insulin at the time t+j;
IOB(t+j) represents the amount of insulin that has not yet worked in the body at the time t+j.

25. According to the closed-loop artificial pancreas insulin infusion control system of claim 1, wherein,
any two of the detection module, the program module and the infusion module are connected to each other, and are configured to form a single part whose attached position on a skin is different from the third module.

26. According to the closed-loop artificial pancreas insulin infusion control system of claim 1, wherein,
the detection module, the program module and the infusion module are connected together, and are configured to form a single part which is attached on only one position on a skin.
